(19) United States
(12) Patent Application Publication
(10) Pub. No.: US 2026/0248802 A1
(43) Pub. Date: Aug. 27, 2026

(54) DOSAGE OF BALOXAVIR MARBOXIL FOR PEDIATRIC PATIENTS

(71) Applicants: Hoffmann-La Roche Inc., Little Falls, NJ (US); Shionogi & Co., Ltd., Osaka (JP)

(72) Inventors: Stefan De Buck, Basel (CH); Sylvie RETOUT, Basel (CH); Toshihiro WAJIMA, Osaka (JP); Toru ISHIBASHI, Osaka (JP)

(21) Appl. No.: 19/385,628

(22) Filed: Nov. 11, 2025

Related U.S. Application Data
(63) Continuation of application No. 16/991,451, filed on Aug. 12, 2020.
(60) Provisional application No. 62/885,989, filed on Aug. 13, 2019.

(51) Int. Cl. A61K 31/5383; A61K 9/16; A61K 47/02; A61K 47/14; A61K 47/22; A61K 47/26; A61K 47/32; A61K 47/38; A61P 31/16

(52) U.S. Cl. CPC A61K 31/5383; A61K 9/1694; A61K 47/02; A61K 47/14; A61K 47/22; A61K 47/26; A61K 47/32; A61K 47/38; A61P 31/16

(57) ABSTRACT

The present invention relates to a method for treating an influenza virus infection, wherein said method comprises administering an effective amount of a compound to a patient having an influenza virus infection, wherein the compound has one of the formulae (I) and (II), as set forth herein, or is a pharmaceutically acceptable salt thereof, and wherein dosages set forth herein are used.

### CROSS-REFERENCE OF RELATED APPLICATIONS

This application is a continuation application of Unites States Patent application Ser. No. 16/991,451, filed on Aug. 12, 2020, which claims the benefit of priority to U.S. Provisional Patent Application No. 62/885,989, filed on Aug. 13, 2019, the contents of each of which are incorporated by reference herein in their entirety for all purposes.

### SEQUENCE LISTING

The instant application contains a Sequence Listing which has been submitted electronically in XML format and is hereby incorporated by reference in its entirety. Said XML copy, created on Nov. 11, 2025, is named 050045-554C01US_sequence_listing.xml and is 4,142 bytes in size.

### BACKGROUND

Influenza is an acute respiratory infectious disease caused by a virus of the orthomyxovirus family. Two forms are known to infect humans, influenza A and B. These viruses cause an acute febrile infection of the respiratory tract after an incubation period of 1 to 4 days, characterized by the sudden onset of fever, cough, fatigue, headache, and myalgia. Annual influenza epidemics are thought to result in between 3 to 5 million cases of severe illness, and between 250,000 and 500,000 deaths every year around the world (WHO fact sheet 211: influenza (seasonal). 2018).

Although the condition is usually self-limiting in healthy adults, it can be associated with substantial morbidity and occasional mortality in children, the elderly, and the immunocompromised (Paules, Subbarao. Lancet 2017; 390: 697-708). Children play a central role in the dissemination of influenza in the community by virtue of their relative serosusceptibility and consequently higher illness attack rates. In addition to the acute illness, young children are at particular risk of secondary bacterial infections. Such secondary bacterial infections lead to poor prognosis, particularly in children. Other serious complications can also develop, including cardiac and neurological complications. Children develop more severe disease compared with adults, with higher hospitalization rates particularly in children aged <5 years (Rotrosen, Neuzil. Pediatr Clin North Am 2017; 64: 911-36). Although NA inhibitors, such as oseltamivir, zanamivir, and peramivir, can be used for the treatment of pediatric patients at present, more convenient and potent anti-influenza virus drugs without restriction of use are needed for the following reasons: 1) zanamivir is not licensed for treatment of influenza in very young children due to the difficulty with inhalation in this group (<5 or 7 years of age, depending on country), 2) peramivir needs to be intravenously administered, and 3) oseltamivir requires twice daily (BID) dosing orally for 5 days. In addition, the efficacy of currently marketed antivirals against preventing complications in pediatric patients has not been demonstrated.

### SUMMARY OF THE DISCLOSURE

The present invention relates to a method for treating influenza, wherein said method comprises administering an effective amount of a compound to a pediatric patient in need thereof.

In one aspect, provided is a method for treating an influenza virus infection, wherein said method comprises administering an effective amount of a compound to a patient having an influenza virus infection, wherein the compound has one of the formulae (I) and (II), as set forth herein, or is a pharmaceutically acceptable salt thereof, and wherein the following dosage is used: (i) in a patient that is younger than 1 year: (a) if the patient is younger than 4 weeks, then the effective amount is 0.8-1.2 mg/kg body weight, preferably about 1 mg/kg body weight; (b) if the patient is 4 weeks or older but younger than 3 months, then the effective amount is 0.8-1.2 mg/kg body weight, preferably about 1 mg/kg body weight; (c) if the patient is 3 months or older but younger than 12 months, then the effective amount is 1.8-2.2 mg/kg body weight, preferably about 2 mg/kg body weight; (ii) in a patient that is 1 year or older but younger than 12 years: (a) if the patient has a body weight of less than 20 kg, then the effective amount is 1.8-2.2 mg/kg body weight, preferably about 2 mg/kg body weight; or (b) if the patient has a body weight of 20 kg or more, then the effective amount is 35-45 mg, preferably about 40 mg.

### INCORPORATION BY REFERENCE

The content of all documents cited herein above and below is incorporated by reference in its entirety. In particular, all publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The present invention is further described by reference to the following non-limiting figures and examples. The Figures show:

FIGS. 1A-1C: Simulated total drug exposure for three different dosing regimens in pediatrics (Non-Asian, Age 1-12 year olds). Bottom and top of the boxplot represent 25^{th} and 75^{th} percentile; middle line in the box represents 50^{th} percentile; lower and upper whisker represent 10^{th} and 90^{th} percentile. Note: for ease of simulations of regimen 2, the weight at which bodyweight-based dosing converts to flat dosing was 26.6 kg.

FIGS. 2A-2C: Simulated peak drug exposure for three different dosing regimens in pediatrics (Non-Asian, Age 1-12 year olds). Bottom and top of the boxplot represent 25^{th} and 75^{th} percentile; middle line in the box represents 50^{th} percentile; lower and upper whisker represent 10^{th} and 90^{th} percentile. Note: for ease of simulations of regimen 2, the weight at which bodyweight-based dosing converts to flat dosing was 26.6 kg.

FIGS. 3A-3C: Simulated drug exposure at 24 hours after dosing for three different dosing regimens in pediatrics (Non-Asian, Age: 1-12 year olds). Bottom and top of the boxplot represent 25^{th} and 75^{th} percentile; middle line in the box represents 50^{th} percentile; lower and upper whisker represent 10^{th} and 90^{th} percentile. Note: for ease of simulations of regimen 2, the weight at which bodyweight-based dosing converts to flat dosing was 26.6 kg.

FIGS. 4A-4C: Simulated drug exposure at 72 hours after dosing for three different dosing regimens in pediatrics (Non-Asian, Age: 1-12 year olds). Bottom and top of the boxplot represent 25^{th} and 75^{th} percentile; middle line in the box represents 50^{th} percentile; lower and upper whisker represent 10^{th} and 90^{th} percentile. Note: for ease of simulations of regimen 2, the weight at which bodyweight-based dosing converts to flat dosing was 26.6 kg.

FIGS. 5A-5C: Simulated total drug exposure for three different dosing regimens in pediatrics (Non-Asian, Age: <1 year old). Bottom and top of the boxplot represent 25^{th} and 75^{th} percentile; middle line in the box represents 50^{th} percentile; lower and upper whisker represent 10^{th} and 90^{th} percentile. Grey box with rounded edges indicates nearly identical match with adult exposures in this model.

FIGS. 6A-6C: Simulated peak drug exposure for three different dosing regimens in pediatrics (Non-Asian, Age: <1 year olds). Bottom and top of the boxplot represent 25^{th} and 75^{th} percentile; middle line in the box represents 50^{th} percentile; lower and upper whisker represent 10^{th} and 90^{th} percentile.

FIGS. 7A-7C: Simulated drug exposure at 24 hours after dosing for three different dosing regimens in pediatrics (Non-Asian, Age: <1 year olds). Bottom and top of the boxplot represent 25^{th} and 75^{th} percentile; middle line in the box represents 50^{th} percentile; lower and upper whisker represent 10^{th} and 90^{th} percentile.

FIGS. 8A-8C: Simulated drug exposure at 72 hours after dosing for three different dosing regimens in pediatrics (Non-Asian, Age: <1 year olds). Bottom and top of the boxplot represent 25^{th} and 75^{th} percentile; middle line in the box represents 50^{th} percentile; lower and upper whisker represent 10^{th} and 90^{th} percentile.

FIG. 9: Canadian Acute Respiratory Illness and Flu Scale (CARIFS) Questionnaire.

FIG. 10: The powder X-ray diffraction pattern of the crystal of compound I of Example 6.

### DETAILED DESCRIPTION

Baloxavir marboxil is a compound discovered by Shionogi & Co., Ltd. that exerts antiviral effects against influenza. Baloxavir marboxil (also referred to as S-033188, Shionogi Compound Identification Number) is a pro-drug that is converted to the active form baloxavir (also referred to as S-033447, Shionogi Compound Identification Number) in the blood, liver, and small intestine through a metabolic process called hydrolysis. Baloxavir marboxil acts on the cap-dependent endonuclease, an enzyme specific to influenza viruses, and inhibits viral cap-snatching, thereby suppressing the growth of influenza viruses.

Baloxavir marboxil has been tested in several clinical trials. However, it is commonly known that the results of a given clinical trial cannot be simply transferred to the response of any patient to the pharmaceutical compound. More specifically, there are several factors that may have significantly influenced the outcome of the clinical trial, such as the patient population (e.g. adults, pediatrics, elderly, ethnicity) and the dosing regimen.

For example, it is known that the results of clinical trials on adults cannot be transferred to pediatric patients. To find a dose that produces the desired therapeutic effect and at which no side effects occur must be determined separately in minors, even if suitable doses are known for adults. Finding a dose which is particularly suitable for minors is very important because a young organism processes drugs very differently from an adult. Newborns, for example, only degrade drugs slowly because the liver and kidneys are not yet mature. Children over two years of age, on the other hand, have a faster metabolism and their bodies sometimes excrete the substances more quickly. Furthermore, medicaments which are usually harmless in adults can be dangerous for children. For example, the compound acetylsalicylic acid (ASS), which is commonly used by adults suffering from pain or fever, can trigger the life-threatening Reye syndrome in children, which can severely damage the brain and the liver. Therefore, clinical trials on adults cannot be used for determining whether a given compound can be used in minors, even less for finding a suitable dose of the medicament in minors, children and newborns.

Indeed, the oral clearance of baloxavir (CL/F) was influenced by bodyweight. The lower bodyweight, the higher CL/F. This relationship suggest that CL/F will increase with age. In a population pharmacokinetic (PK) analysis based on a Japanese pediatric trial (1618T0822), the CL/F relationship was defined as follow: CL/F=3.05*(Bodyweight/24.3)^{0.632}. A similar impact of bodyweight was observed on the baloxavir apparent volume of distribution. Similarly, a lower central volume of distribution was observed in patients with lower bodyweight (Vc=105*(Bodyweight/24.3)^{1.03}). Due to this impact of bodyweight on PK of baloxavir, the dose which is used in adults cannot simply be extrapolated for obtaining optimal drug exposure in pediatric patients matching drug exposure of adults in terms of both total area under the plasma concentration-time curve (AUC) and plasma concentration 72 hours after dosing (C₇₂).

Two phase III clinical trials have been conducted for testing baloxavir marboxil in pediatric patients from 6 months to 12 years of age in Japan (studies 1618T0822 and 1705T0833). All participants of these studies had Asian heritage (Japanese) and the highest administered dosage was 40 mg. In the first study 1618T0822 (also called T0822) tablets of 10 mg and 20 mg were used. Patients were dosed per bodyweight as follows: ≥40 kg: 40 mg dose (n=8), 20 kg-40 kg: 20 mg dose (n=66), 10 kg-20 kg: 10 mg dose (n=31), 5 kg-<10 kg: 5 mg (n=2). In the second paediatric study in Japanese patients 1705T0833 (also called T0833) baloxavir marboxil 2% granules were administered to paediatric subjects weighing less than 20 kg and less than 12 years of age. 33 patients aged between 0 and 6 year-old were included in this study. 6 were less than 1 year, 13 between 1 and 3, and 14 were 3 years or older. 12 subjects had bodyweight lower than 10 kg, and 21 had bodyweight lower than 20 kg.

Concern that ethnic differences may affect the medication's safety, efficacy, dosage and dose regimen in a new region has limited the willingness to rely on foreign clinical data. Indeed, it is known that the varieties in metabolism of persons having a different ethnicity are associated with interethnic variation in drug pharmacokinetics (Kim, The Journal of Clinical Pharmacology 44.10 (2004): 1083-1105). It was also known that such interethnic variations particularly exist between Asians (such as Japanese persons) and white persons (e.g. Caucasians), and can lead to differences in efficacy and toxicity of a given drug (Kim, The Journal of Clinical Pharmacology 44.10 (2004): 1083-1105). The ICH (International Council for Harmonisation of Technical Requirements for Pharmaceuticals for Human Use) guidelines E5(R1) defined ethic factors and their inclusion in multiregional clinical trials (see IHC guideline E5(R1) of Feb. 5, 1998 including corrections of Mar. 11, 1998). For example, the ICH guideline E5 makes clear that clinical data which has been obtained with patients having a particular heritage cannot simply be transferred to patients having a different heritage. The reason is that several medical compounds are sensitive to ethnic factors, which means that ethnic factors (such as genetic polymorphisms) have significant impact on safety, efficacy, or dose response of the compounds. There are several examples where the ethnic heritage considerably influenced the response to a drug (Bjornsson, The Journal of Clinical Pharmacology 43.9 (2003): 943-967). Indeed, interethnic variability in pharmacokinetics can cause unexpected outcomes such as therapeutic failure, adverse effects, and toxicity in subjects of different ethnic origin undergoing medical treatment (Kim, The Journal of Clinical Pharmacology 44.10 (2004): 1083-1105). For example, it is known in the art that a particular splicing polymorphism in the enzyme UGT2B10 which is common in African populations can greatly increase drug exposure (Fowler, Journal of Pharmacology and Experimental Therapeutics 352.2 (2015): 358-367). This UGT2B10 splice site mutation is almost unrepresented in Caucasians (Fowler, Journal of Pharmacology and Experimental Therapeutics 352.2 (2015): 358-367). Similarly, a clinical study on the treatment of gastric cancer with bevacizumab showed regional differences in efficacy outcomes (Ohtsu, J Clin Oncol 29.30 (2011): 3968-3976).

In the treatment of influenza it is of high importance to use an appropriate dosage of the anti-influenza drug. For example, a dosage too low can lead to the occurrence of treatment-resistant viruses (e.g. viruses having the 138 amino acid substitution). A dosage too low can further lead to rebound of virus titer or double-peak fever. Therefore, in the treatment of influenza it is of high importance to use a dose of the anti-influenza drug which is as high as necessary for obtaining a fast therapeutic response by avoiding overdose.

As described above, baloxavir marboxil has been tested in various clinical studies in adults as well as in a small number of clinical studies in Asian pediatric patients. However, as also explained above, these data cannot simply be transferred to non-Asian pediatric patients. In addition, as also explained above, usage of the correct dose is of high importance in the treatment of influenza.

Thus, the technical problem underlying the present invention is the provision of an improved dosage of baloxavir marboxil for pediatric patients.

The technical problem is solved by provision of the embodiments as characterized in the claims.

Accordingly, the present invention relates to a method for treating an influenza virus infection, wherein said method comprises administering an effective amount of a compound to a patient having an influenza virus infection, wherein the compound has one of the following formulae (I) and (II):

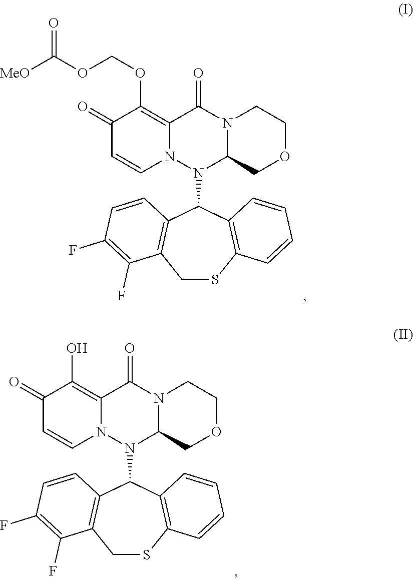 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or is a pharmaceutically acceptable salt thereof,
and wherein the following dosage is used:
(i) in a patient that is younger than 1 year:
   (a) if the patient is younger than 4 weeks, then the effective amount is 0.8-1.2 mg/kg body weight, preferably about 1 mg/kg body weight;
   (b) if the patient is 4 weeks or older but younger than 3 months, then the effective amount is 0.8-1.2 mg/kg body weight, preferably about 1 mg/kg body weight;
   (c) if the patient is 3 months or older but younger than 12 months, then the effective amount is 1.8-2.2 mg/kg body weight, preferably about 2 mg/kg body weight;
(ii) in a patient that is 1 year or older but younger than 12 years:
   (a) if the patient has a body weight of less than 20 kg, then the effective amount is 1.8-2.2 mg/kg body weight, preferably about 2 mg/kg body weight; or
   (b) if the patient has a body weight of 20 kg or more, then the effective amount is 35-45 mg, preferably about 40 mg.

As mentioned above, in the treatment of influenza, a dosage too low can affect the occurrence of treatment-resistant viruses (e.g. viruses having the 138 amino acid substitution), and can further lead to rebound of virus titer and double-peak fever. The dosage of the present invention preferably reduces the occurrence of treatment-resistant viruses (e.g. viruses having the 138 amino acid substitution) as compared to pediatric baloxavir marboxil dosages of the prior art. In addition, the dosage of the present invention preferably reduces the occurrence of viral rebound as compared to pediatric baloxavir marboxil dosages of the prior art. As used herein the term “viral rebound” means: For observed time points after administration of the compound, influenza virus titer [log 10(TCID50/mL)] at a certain time point is equal to 0.6 or 0.6 greater than that atjust before time point. Furthermore, the dosage of the present invention preferably reduces the occurrence of double-peak fever as compared to pediatric baloxavir marboxil dosages of the prior art. The dosage of the present invention may further shorten the time to alleviation of influenza illness and/or resolution of fever as compared to pediatric baloxavir marboxil dosages of the prior art.

As shown in the appended Examples, there was a clear difference in the median time to cessation of viral shedding between baloxavir (24 hrs) and oseltamivir (76 hrs). These data indicate that baloxavir-treated patients are no longer infective after a median time of about 1 day compared to about 3 days in oseltamivir-treated patients. Accordingly, the dosage of the present invention advantageously reduces transmission of influenza. More specifically, the dosage of the present invention preferably reduces transmission of the influenza virus of a patient who received the dosage of the present invention as compared to patients who received oseltamivir. The patient is a pediatric patient which is newborn or older but younger than 12 years, e.g., 1 year or older but younger than 12 years.

As discussed above, in the treatment of influenza it is of high importance to use an appropriate dose of the anti-influenza drug which is as high as necessary for preventing occurrence of treatment-resistant viruses or viral rebound, however, by avoiding overdose. Predicting the suitable dose of a drug for a desired patient group is an important measure for ensuring that the drug is administered to the patients in a sufficient dose to obtain the desired therapeutic effect while avoiding overdose. Such predictions can be performed in silico by using a suitable descriptive or mechanistic model. Of course, modeling techniques do not provide complete certainty that a given patient shows the desired response to the tested drug. However, the same holds true for every clinical testing. Favorable results from biochemical or cell-based assays which test the effects of a drug as well as animal experiments or even clinical trials involving patients can only increase the probability that the drug shows the desired therapeutic effects in the subsequently treated patients. For example, early phase studies usually have a small sample size or may be biased for an unknown reason, which may lead to an incorrect assessment of the physiological effects of the drug at issue. It is nearly impossible to absolutely proof that a medicament will (always) show the desired therapeutic effect in the intended patient group without leading to any unwanted side-effects. As mentioned, all possible methods for verifying the physiological effects of a drug can only increase the possibility that the drug will lead to this particular physiological effect in the later on treated patient. As explained above, predicting a suitable dose of a drug by in silico modeling is one of these models, which is particularly useful for establishing a suitable dose for a new patient group.

In the context of the present invention comprehensive model simulations to predict a suitable dose of baloxavir marboxil in pediatric patients (preferably non-Asian pediatric patients) have been performed. The model used for the simulations was developed by considering previous studies conducted in Japanese pediatric patients. The model integrates both patient's demographics characteristics and drug PK characteristics in the studied population. Baloxavir plasma concentrations after various dosing regimen can then be simulated in pediatric patients based on patient characteristics such as age or bodyweight. Consequently, this model advantageously provides the basis for a suitable dose of baloxavir marboxil in pediatric patients, preferably non-Asian (such as white) pediatric patients, which, in all likelihood, ensures baloxavir plasma exposures comparable to exposures in adult patients and appropriate pharmacologic effect in the treatment of influenza by avoiding potential side-effects.

More specifically, in the context of the present invention suitable doses for non-Asian (e.g. white, such as Caucasian) pediatric patients were determined using a modeling and simulation approach. Based on a model developed in Japanese pediatric patients, plasma concentrations of baloxavir (S-033447) pharmacokinetics in a non-Asian (e.g. white, such as Caucasian) pediatric population were simulated for different dosing regimen. More specifically, a population pharmacokinetic analysis had been performed in Japanese pediatric populations by using unpublished pharmacokinetic data obtained in a phase III study involving pediatric patients in Japan (1618T0822); the suitable dose of baloxavir marboxil for non-Asian pediatric patients was then obtained by simulating non-Asian pediatric drug exposure after several different dosing regimen, the ones matching the best adult exposures were then selected. In particular, the simulation of pediatric drug exposure was performed as described in the following:

With respect to non-Asian pediatric patients that are younger than 1 year, simulations were performed for 1,000 patients for each age in months for <2-year old infants (26,000 patients in total). Thus, several sets of 1000 patients were conducted. For instance, 1000 between 0 and 1 month, 1000 between 1 and 2 months, . . . for a total of 26000 simulations (i.e. 26×1000). For patients that are between 1 and 12 years old, simulation of non-Asian pediatric drug exposure was performed for 1,000 patients for every 5-kg body weight for 10- to 60-kg pediatric patients (26,000 patients in total).

In both cases, various dosing regimens were evaluated with respect to their ability to match adult drug exposure in terms of area under the plasma concentration-time curve (AUC), maximum plasma concentration (Cₘₐₓ), plasma concentration 24 hours after dosing (C₂₄; acceptable time window: 20 to 28 hours), and 72 hours after dosing (C₇₂). The optimal dose and appropriate age and bodyweight cut-off were based on a comparison of the simulated drug exposures with those obtained in the phase III study (1601T0831) for patients receiving 40 mg baloxavir marboxil (body weight <80 kg) and patients receiving 80 mg baloxavir marboxil (body weight ≥80 kg), those obtained in the pediatric phase III study (1618T0822), and those obtained in the phase I thorough corrected QT interval (QTc) study (1527T0816) for patients receiving 80 mg baloxavir marboxil.

With respect to patients that are younger than 1 year simulations showed that optimal exposure matching to adults in terms of both total (AUC) and sustained (C₇₂) drug exposure was achieved with 2 mg/kg in infants of 3 months and older, and 1 mg/kg in younger infants (4 weeks-3 months) as well as for newborns (0-4 weeks). Accordingly, in patients which are younger than 1 year baloxavir marboxil can be administered according to the infant's age recorded at the time point when the patient is diagnosed as having an influenza virus infection (i.e., 2 mg/kg≥3 months, 1 mg/kg<3 months) to obtain similar exposure of baloxavir (S-033447) to that resulting from the administration of 40 mg or 80 mg baloxavir marboxil (based on the patient's body weight) to adults in the phase III and Japanese pediatric phase III studies.

With respect to patients that are 1 to 12 years old simulations showed that optimal exposure matching to adults in terms of both total (AUC) and sustained (C₇₂) drug exposure was achieved with 2 mg/kg in children weighing less than 20 kg and flat dosing of 40 mg in children weighing ≥20 kg. Accordingly, patients which are 1 to 12 years old baloxavir marboxil can be administered based to the body weight recorded at the time point when the patient is diagnosed as having an influenza virus infection (i.e., 2 mg/kg for patients weighing <20 kg or 40 mg for patients weighing 20 kg) to obtain similar exposure of baloxavir (S-033447) to that resulting from the administration of 40 mg or 80 mg baloxavir marboxil (based on body weight) to adults in phase III and Japanese pediatric phase III studies.

As explained above, the clinical studies which have been conducted with baloxavir marboxil and Asian pediatric patients cannot be simply transferred to non-Asian (e.g. white, such as Caucasian) pediatric patients. Therefore, and in order to provide an optimal dose for children younger than 12 years (and therewith improve the chances of these young patients to recover from an influenza virus infection) an improved dosage schedule for non-Asian (e.g. white) pediatric patients has been developed in accordance with the present invention. Therefore, in accordance with the present invention, the patient to be treated may have the racial designation non-Asian, e.g. “white”. Thus, the invention relates to the herein provided method, wherein the patient is white. The term “white” refers to a person having origins in any of the original peoples of Europe, the Middle East, or North Africa (cf., e.g., US Food and Drug Administration. “Collection of Race and Ethnicity Data in Clinical Trials Guidance for Industry and Food and Drug Administration Staff.” Issued on Oct. 26 (2016)). For example, the white pediatric patient may be Caucasian.

As described above, the guidelines of the ICH make clear that clinical data which has been obtained with patients having a particular heritage cannot simply be transferred to patients having a different heritage. According to the ICH guidelines a clinical trial which has been conducted in one region (like Japan) cannot be transferred to another region (such as Europe or the United States). For example, evaluation of the pharmacokinetics in the three major racial groups most relevant to the ICH regions (Asian, Black, and Caucasian) is critical to the registration of medicines in the ICH regions. With respect to baloxavir marboxil, clinical trials have been conducted with pediatric patients in Japan. The present invention is based on the finding of an optimal baloxavir marboxil dose for non-Asian (e.g. white, such as Caucasian) pediatric patients. Accordingly, in accordance with the present invention the patient has preferably a non-Asian heritage and is not living in Asia. Thus, in the present invention the patient may not have an Asian ethnicity. The term “Asian” refers to a person having origins in any of the original peoples of the Far East, Southeast Asia, or the Indian subcontinent, including, for example, Cambodia, China, India, Japan, Korea, Malaysia, Pakistan, the Philippine Islands, Thailand, and Vietnam. (cf., e.g., US Food and Drug Administration. “Collection of Race and Ethnicity Data in Clinical Trials Guidance for Industry and Food and Drug Administration Staff.” Issued on Oct. 26 (2016)). For example, the patient may not be Japanese.

Thus, it is preferred that the patient does not have an Asian (e.g. Japanese) ethnicity and does not live in Asia (e.g. Japan). As mentioned above, a clinical trial with baloxavir marboxil has been conducted with Japanese pediatric patients (studies 1618T0822 and 1705T0833). However, in these studies the efficacy of a maximum of 1 mg/kg body weight of baloxavir marboxil was used in patients aged 6 months to <12 years. Thus, these Japanese clinical trials are significantly different from the dosages which are provided herewith, since in the context of the present invention the patients can be younger than 6 months, and/or receive 2 mg/kg body weight of baloxavir marboxil. In addition, as explained above, the results of clinical trials cannot be directly transferred from one ethnicity to another. Therefore, the clinical trials which have been conducted in Japan with Asian pediatric patients (studies 1618T0822 and 1705T0833) cannot be directly transferred to non-Asian (e.g. white, such as Caucasian) pediatric patients. As mentioned above, the dosages provided with the present invention are optimized dosages for non-Asian, (e.g. white, such as Caucasian) pediatric patients. Therefore, in the present invention it is preferred that the pediatric patients are white, e.g. Caucasian. Europeans and “white” Americans are usually referred to as “Caucasians” (Bjornsson, The Journal of Clinical Pharmacology 43.9 (2003): 943-967). Thus, in accordance with the present invention the patient may have Caucasian (i.e. European or “white” American) heritage and may be living in Europe or North-America (e.g. in the United States).

Baloxavir marboxil is mostly administered in the form of tablets. However, tablets have the disadvantages that the acceptability is usually low in pediatric patients, leading to inconsequent drug intake, splitting out of the drug or vomiting the medicine before it takes effect. In addition, newborn and young children are often not able to swallow tablets. Also patients with a nasogastric tube in situ (e.g., intubated patients) are unable to swallow tablets. Therefore, in the context of the present invention the compound may be administered in the form of a suspension of granules. Particularly if the patient is younger than 1 year (i.e. patients as defined under (i), above), or if the patient is 1 year or older and has a body weight of less than 20 kg (i.e. patients as defined under (ii)(a), above) the compound may be administered in the form of a suspension of granules. For example, the granules as described in PCT/JP2019/017146 may be used. It has been shown that such granules (in particular 2% baloxavir marboxil, i.e. S-033188, granules) have bioequivalence with 20 mg baloxavir marboxil (S-033188) tablets (Clinical Study Report, Study No. 1703T081G, Shionogi & Co., Ltd.; 2018). Therefore, in the present invention the granules are preferably 2% baloxavir marboxil (i.e. S-033188) granules.

In the clinical trial with Japanese paediatric patients weighing less than 20 kg (1705T0833) granules have been used as administration form. The finished granule product configuration developed for the Japanese market by Shionogi consists of granules packaged in a sachet. The granules are intended for administration directly into the mouth of the subject. In the context of the present invention the granules are preferably resuspended (e.g. in a bottle) and a specific volume is given orally (e.g. by a syringe). In particular, the granules to be used in the present invention may be reconstituted with water. For example, 2 g of granules, which contain 40 mg of the compound to be used in the present invention (nominal), may be reconstituted with 20 mL water, which corresponds to a final concentration of 2 mg of the compound per millilitre (mL). These resuspended granules can advantageously be administered to children, even to young children (infants) and patients having a nasogastric tube.

The granules for oral suspension may have a composition as shown Table 1.

TABLE 1
Components and composition of baloxavir marboxil granules for oral suspension

| | Nominal | Concentration | | |
|---|---|---|---|---|
| | amount | in Granule | | |
| Component | (mg/bottle) | (%) | Function | Quality Standard |
| Baloxavir Marboxil | 40 | 2 | Active | In-house standard |
| | | | ingredient | |
| Mannitol | 1120 | 56 | Diluent | Ph. Eur./USP/JP |
| Maltitol | 700 | 35 | Diluent | Ph. Eur./NF/JPE |
| Sodium Chloride | 60 | 3 | Taste | Ph. Eur./USP/JP |
| | | | masking | |
| | | | agent | |
| Hypromellose | 6 | 0.3 | Dispersant | Ph. Eur./USP/JP |
| Povidone (K value: | 20 | 1 | Binder | Ph. Eur./USP/JP |
| 25) | | | | |
| Silica, Colloidal | 40 | 2 | Fluidizer | Ph. Eur./NF/JP |
| Anhydrous | | | | |
| Sucralose | 10 | 0.5 | Sweetener | Ph. Eur./NF/JPE |
| Talc | 2 | 0.1 | Lubricant | Ph. Eur./USP/JP |
| Strawberry Flavour | 2 | 0.1 | Flavour | In-house standard |
| Purified Water ^{a} | — | — | Vehicle | Ph. Eur./USP/JP |
| Total Weight ^{b} | 2,000 | 100 | — | — |

^{a} Purified water is removed during manufacturing process.
^{b} An overfill of, e.g. 0.13 g of granules is applied to obtain the targeted maximum extractable volume of 20 mL after reconstitution; fill weight may be adjusted based on assay value for bulk granules.

Bitter taste has been reported in adult clinical studies with baloxavir marboxil and several excipients have been included in the formulation to mask the bitter taste and ensure palatability, such as sodium chloride, sucralose and strawberry flavor. Thus, the granules provided with the present invention have the advantages that they are to be administered in the form of an oral suspension and that the bitter taste of the active compound is masked. Accordingly, these granules improve acceptance of the compound in pediatric patients, which contributes to the achievement of the therapeutic effect. Indeed, in clinical trials wherein baloxavir marboxil was administered to pediatric Japanese patients (i.e. studies 1618T0822 and 1705T0833) the most common adverse event was vomiting. Although the vomiting was considered to be not related to the study drug by the investigators, reducing or avoiding vomiting which is induced by the administration form can provide a therapeutic benefit. In addition, the oral suspension provides flexibility to more precisely implement weight-based dosing.

As dosing device an oral dosing syringe or an oral dosing cup (both volumetric) may be used to provide the sufficient degree of accuracy to deliver the recommended doses of the compound to be used in the present invention (e.g. baloxavir marboxil). For example, a 3 mL oral dosing syringe that could be used in infants typically includes volumetric demarcations in tenths of a milliliter, which would be adequate to deliver accurate doses. Alternatively, a 10 mL oral dosing syringe may be used.

Examples for dosages which may be used are shown below in Table 2.

TABLE 2
Examples for age/weight dependent dosing

| | | dose volume of | | | | |
|---|---|---|---|---|---|---|
| | | 2% suspension | | | | |
| Age group | weight (kg) | (mL) | dose regime | | Age | |
| Infants | 2 | 1 | | | | |
| (i.e. young | 3 | 1.5 | | | | |
| children) | 4 | 2 | | | | |
| | 5 | 2.5 | 1 | mg/kg | <3 | months |
| | 6 | 6 | 2 | mg/kg | | |
| | 7 | 7 | | | | |
| | 8 | 8 | | | | |
| | 9 | 9 | | | | |
| | 10 | 10 | | | <12 | months |
| Children | 11 | 11 | 2 | mg/kg | | |
| | 12 | 12 | | | | |
| | 13 | 13 | | | | |
| | 14 | 14 | | | | |
| | 15 | 15 | | | | |
| | 16 | 16 | | | | |
| | 17 | 17 | | | | |
| | 18 | 18 | | | | |
| | 19 | 19 | | | | |
| | <20 | 20 | | | | |
| | ≥20 | 20 | 40 | mg (flat dose) | | |

The dosing shown in Table 2, above, is merely an example. For example, the dosage of patients as defined in the inventive method under item (i), above, (i.e. patients who are younger than 1 year) is performed according to their age (e.g., 1 mg/kg for patients who are younger than 3 months; and 2 mg/kg for patients who are 3 months or older but younger than 12 months). For example, a child who is younger than 3 months and has a body weight of 6 kg would receive about 1 mg/kg of the compound.

As described above, the compound to be used in the present invention can be administered in the form of a suspension of granules. Such granules for oral suspension can be reconstituted with water to provide the desired dose. However, according to the present invention a patient who is 1 year old or older and has a body weight of 20 kg or more (i.e. the patient as defined in item (ii)(b), above) receives a 40 mg flat dose of the compound. This 40 mg dose is preferably administered in the form of a tablet. For example, the 40 mg dose may be administered in the form of a film-coated tablet.

However, the invention is not limited to any specific route of administration of the compound to be used herein. All possible routes of administration that the attending physician deems useful or necessary are within the scope of the present invention. For example, the compound may be administered oral, rectal, nasal, topical, intradermal, as aerosol, vaginal, or parenteral, such as intramuscular, intravenous, subcutaneous, intraarterial, or intracardial. It is preferred that the compound is orally administered. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems. However, it is preferred in the present invention that the compound is orally administered. It is envisaged in the present invention that the compound is given as a single dose.

As indicated above, particularly for the patients as defined under (i) and (ii)(a), above, it is preferred that the compound to be used in the present invention is in the form of the granules for suspension, and is administered as single oral dose, or as single dose which is administered via a nasogastric tube. For example, the granules comprising baloxavir marboxil as described above can be reconstituted with water to provide the desired dose in a suspension. If the patient is ≥1 year but <12 years and has a body weight of 20 kg or more (i.e. the patient as defined in (ii)(b), above), then the effective amount of the compound to be used herein is 35-45 mg, preferably about 40 mg. For these patients administration of two 20 mg tablets or one 40 mg tablet as single oral dose is preferred.

Children usually reach 20 kg with the age of about 3 to 8 years, mostly between 5 and 6 years. Therefore, in the context of the present invention the patient as defined in (ii)(a), above (i.e. the patient that is ≥1 year but <12 years, and has a body weight of <20 kg) may have an age between 1 and 8 years, e.g. between 1 and 6 years. For example, the patient as defined under (ii)(a), above, may be 1 year old or older but younger than 5 years. 1 year old children have usually a weight between 7 and 13 kg. Therefore, the patient as defined in (ii)(a) may have a body weight which is about 7 kg or more, e.g. about 11 kg or more.

In the present invention the patient as defined under (ii)(b), above (i.e. the patient that is ≥1 year but <12 years, and has a body weight of ≥20 kg) may be 5 years old or older but younger than 12 years. In addition or alternatively, the patient as defined under (ii)(b), above, may have a body weight which is less than 40 kg. According to the present invention a patient that is 1 year or older but younger than 12 years and has a body weight of 20 kg or more is administered with an effective amount of the compound to be used in the invention which is 35-45 mg, preferably about 40 mg. It is preferred that the compound is administered to this patient in an amount which is more than 1 mg/kg body weight (e.g. 1.5-2 mg/kg body weight).

In accordance with the present invention a comprehensive simulation has been performed in order to find optimal doses of baloxavir marboxil for pediatric patients, particularly non-Asian (e.g. white such as Caucasian) pediatric patients. This simulation shows that the regimen of the present invention matches adult drug exposure optimally in terms of both total drug exposure as well as drug levels up to 72 hours after dosing, especially in pediatrics with a body weight less than 25 kg. Therefore, the patient as defined in item (ii)(b), above (i.e. the patient having a body weight of 20 kg or more) has preferably a body weight which is less than 25 kg.

In the present invention the patient may be healthy except for the influenza virus infection. The influenza virus may have no substitution in at least one of the genes selected from the viral acidic polymerase (PA) gene, the viral basic polymerase 1 (PB1) gene, and the viral basic polymerase 2 (PB2) gene. For example, the influenza virus may have no substitution in all of these genes. In a preferred aspect of the present invention the influenza virus strain does not carry an I38X mutation, such as the I38T mutation, in the viral acidic polymerase (PA) protein. The I38T mutation is commonly known in the art and described, e.g., in Omoto, Scientific reports 8.1 (2018): 9633. Thus, it is preferred that the influenza virus stain does not carry an I38T mutation in the viral acidic polymerase (PA) protein. The I38T substitution is a mutation in the viral acidic polymerase (PA) protein of some mutated influenza A strains. The sequence of the PA protein of an influenza A virus having the I38T mutation is shown in SEQ ID NO:1.

Thus, in a preferred aspect of the present invention the influenza virus strain does not comprise a PA protein having the sequence of SEQ ID NO:1. It is also preferred that the influenza virus strain does not comprise a PA protein having a sequence which has at least 80%, preferably at least 90%, more preferably at least 95%, at least 96%, at least 97%, at least 98%, or at least 99% sequence identity to the sequence of SEQ ID NO:1 and comprising a substitution (e.g. an I to T substitution) at the position corresponding to position 138 of SEQ ID NO:1. A fraction of the PA protein of an influenza A virus comprising the I38T mutation is shown in SEQ ID NO:2. Thus, in a preferred aspect of the present invention the influenza virus strain does not comprise a PA protein comprising the sequence as shown in SEQ ID NO:2.

In one aspect of the present invention an influenza virus infection is present if the influenza virus can be detected. The influenza virus may be detected via PCR. In addition or alternatively the influenza virus may be detected by using an influenza test kit. Rapid Influenza Diagnostic Test (RIDTs) based on immunologic detection of viral antigen in respiratory secretions offer point of care (on-site) tests with results available within 30 minutes. Thus, a RIDT may be used for detecting the influenza virus. RIDTs can identify the presence of influenza A or B viral nucleoprotein antigens and display the result in a qualitative way (positive vs. negative) (Ali T, Clin Infect Dis. 2004 Mar. 1; 38(5):760-2). RIDT assays are ELISA based assays which are less accurate than PCR, but have the advantage that they are cheaper and faster.

The influenza virus infection may further be detected by using the Roche Cobas® Liat® point of care (POC) polymerase chain reaction (PCR) system (Chen, Eur J Microbiol Immunol (Bp). 2015; 5(4):236-245). The Cobas® Liat® system enables rapid and accurate diagnosis of influenza A or B nasopharyngeal swab specimens. The system comprises the Cobas® Liat® Analyzer and the Cobas® Influenza A/B assay. The detection of the influenza virus may also be carried out by using a PCR-based molecular test (Prodesse ProFlu+assay, Chen, Eur J Microbiol Immunol (Bp). 2015; 5(4):236-245) or the Alere i Influenza A & B rapid PCR system (Merckx, Ann Intern Med. 2017; 167(6):394-409).

The influenza virus infection may also be detected by virus culture techniques, which involve inoculation of clinical specimens onto cell culture lines. By using this method, over 90% of positive cultures can be detected within 3 days of inoculation (Newton, Journal of Clinical Microbiology 40.11 (2002): 4353-4356). The influenza virus infection may also be detected via molecular diagnostic tests, which use detection of viral nucleic acids in clinical specimens to achieve greater sensitivity than cell culture and in addition allow detection of virus in samples that have lost viability.

As indicated above, the influenza virus infection may be detected via polymerase chain reaction (PCR) assays, which allow both qualitative and quantitative assessments in addition to rapid subtyping of the virus. The PCR detection and quantification of the influenza virus is commonly known in the art. For Example, real-time reverse transcription PCR (RT-PCR) amplification of the influenza matrix gene may be employed as the method for determining the presence or absence, or the quantity of influenza RNA. Influenza virus RNA extraction and purification is a routine technique and can, e.g., be performed by using a MagNA Pure LC 1.0 or 2.0 isolation station (Roche Applied Science, product #05197686001). To perform the test, nucleic acids are extracted from swab specimen aliquots using the MagNA Pure LC isolation station and the MagNA Pure LC nucleic acid extraction kit according to the manufacturer's instructions (Roche Applied Science). Reverse transcription and amplification reactions can be set up using Taqman Fast Virus Mastermix. During clinical analysis, a 4 point (low, middle and high) influenza A and B standard curve with known virus particles/ml can be used as control and can accompany every run. To monitor the whole process from isolation to real-time detection, a universal internal control, the Phocine Distemper Virus (PDV), may be added to each isolate. In addition, to monitor contamination in every isolation a No Amplification Control (NAC) may be included for every PCR mix that is made. The positive controls must give a positive signal that lies between specified action limits. If the value of the positive control lies outside the action limit, all samples tested with the same PCR mix need to be retested. If the negative control gives a positive signal for influenza, all samples run with the same PCR mix need to be retested. The output of the influenza RT-PCR assay is what is known as a Cycle threshold, or Ct value and a Ct value is recorded for each test. The Ct values are converted to quantitative virus particles/ml values with the standard curves ran concurrent with the samples.

For influenza A positive subjects an influenza A subtype PCR assay can also be performed. More specifically, for influenza A positive subjects, sub-typing can be performed directly from a subject's swab sample using a real time RT-PCR assay. RNA can be isolated from clinical isolates as described above using the Roche MagNA Pure Total Nucleic Acid kit, and can be amplified using a one-step RT-PCR with influenza A-subtype specific primers. Further methods for the detection of particular influenza virus subtypes including suitable primer sequences are commonly known in the art, and described, e.g., in the “WHO information of the molecular detection of influenza viruses” of July 2017.

Serological tests, such as complement fixation and haemagglutination inhibition, can be used to establish retrospectively a diagnosis of an influenza virus infection. Because individuals may have been previously infected with influenza viruses, paired serum specimens, consisting of an acute serum specimen and a convalescent serum specimen, obtained 28 days later, may be used for testing.

Most cases of influenza are diagnosed based on compatible clinical symptoms and seasonal epidemiology. Thus, also the presence of at least one symptom of influenza indicates that an influenza virus infection is present. Therefore, in accordance with the present invention the patient may be diagnosed as having an influenza virus infection:
(i) due to the presence of fever of 38° C. or more (tympanic temperature); and at least one respiratory symptom, preferably cough and/or nasal congestion; and/or
(ii) by using an influenza test kit.

Influenza viruses cause an acute febrile infection of the respiratory tract characterized by the sudden onset of fever, cough, fatigue, headache, and myalgia. The principal clinical presentation of influenza disease is essentially common between adults and children, characterized by rapid onset fever and cough, symptoms generally accepted to be directly consequential to viral replication and the host immune response (innate especially) to viral replication. Beyond the cardinal symptoms of flu, gastrointestinal symptoms, such as vomiting and/or diarrhea (Minodier, Virology Journal 12.1 (2015): 215) can be more common in infants and young children than in adults, and children, particularly those aged <5 years, may have higher maximum temperatures and higher hospitalisation rates than adults (Paules and Subbarao, 2017, Rotrosen and Neuzil, 2017). For example, young children usually have temperatures over 39.5° C. and may have febrile seizures (convulsions).

In one aspect of the present invention an influenza virus infection is present if both features apply, i.e. the influenza virus can be detected, and at least one symptom of an influenza virus infection is present. Said at least one symptom of an influenza virus infection may be a sudden onset of fever, cough, fatigue, headache, and myalgia. The symptoms may further include chills, a sore throat and/or nasal congestion. The symptoms may also include gastrointestinal symptoms. The diagnosis of influenza may also comprise testing whether the body temperature reaches 38° C. to 40° C. within 24 hours from the onset of influenza symptoms (Wright, Fields Virology. 5th ed. (2). Wolters Kluwer Health/Lippincott Williams & Wilkins; 2007. P. 1691-1740; Monto, Arch Intern Med. 2000; 160:3243-3247).

In addition or alternatively, the diagnosis of influenza may be confirmed by all of the following:
(a) Fever ≥38° C. (axillary) in the predose examinations or >4 hours after dosing of antipyretics if they were taken.
(b) At least one of the following general systemic symptoms associated with influenza with a severity of moderate or greater:
   (b)-1 Headache;
   (b)-2 Feverishness or chills;
   (b)-3 Muscle or joint pain;
   (b)-4 Fatigue.
(c) At least one of the following respiratory symptoms associated with influenza with a severity of moderate or greater:
   (c)-1 Cough;
   (c)-2 Sore throat;
   (c)-3 Nasal congestion.
   (c)-4 Influenza A or B infection confirmed by POC PCR testing.

There are three types of influenza viruses: A, B, and C. Types A and B cause widespread outbreaks of influenzal illness nearly every year. Influenza C is associated with sporadic, often asymptomatic infection with little or no mortality and therefore is not of public health concern. In accordance with the present invention the influenza virus may be an influenza A virus or an influenza B virus. For example, the influenza virus may be a type A influenza virus. However, the influenza virus infection may also be a mixed infection involving the influenza A virus as well as the influenza B virus.

The means and methods provided herein are particularly advantageous if the influenza virus strain does not have a resistance against the compound to be used in the present invention. However, the influenza virus strain may have a resistance against other anti-viral drugs (such as peramivir, Ianinamivir, oseltamivir, zanamivir, rimantadine, umifenovir or amantadine). Tests for determining whether a given virus has a resistance against one or more drugs are commonly known in the art and comprise, e.g., the phenotypic resistance assay and the NA-Star assay, which are both described below.

The phenotypic resistance assay may be performed as described in the following: Phenotypic resistance assays (spot/focus reduction assay) can be performed by using the sensitive Virospot detection technology which combines classic virus culture in multi-well microtiter plates and virus-specific immunostaining with automated imaging, detection of infected cells using a CTL Immunospot UV analyzer equipped with Biospot analysis software. The Virospot technology platform determines sensitivity of virus isolates to antiviral drugs measuring IC₅₀/IC₉₀. In brief, the method is based on inoculation of infectious virus on MDCK cell monolayers in 96-well plates in the presence of a drug concentration range. After incubation the cells are fixed and immunostained with virus-specific antibodies followed with TrueBlue substrate and image capture using the UV Analyzer.

The NA-Star assay is particularly useful for determining phenotypic resistance to neuraminidase inhibitors (such as, e.g. oseltamivir), and can be performed as follows: This assay uses a chemiluminescent substrate for highly sensitive detection of neuraminidase enzyme activity. Neuraminidase activity yields a luminescent compound which is quantified by using a reader. Virus neuraminidase activity is determined in the presence of serial dilutions of the neuraminidase inhibitor. Sensitivity to neuraminidase inhibitor is expressed as IC₅₀/IC₉₀ values.

In a preferred aspect of the invention the compound is administered within 96 hours from the time of symptom onset, preferably within 48 hours from the time of symptom onset. For example, in a patient as defined under item (i), above (i.e. a patient that is <1 year) the compound may be administered within 96 hours from the time of symptom onset. In a patient as defined under item (ii), above, (i.e. a patient that is 1 to <12 years) the compound may be administered within 48 hours from the time of symptom onset. The symptom onset may be the time point of the onset of at least one systemic symptom and/or at least one respiratory symptom. Said at least one systemic symptom may be at least one symptom selected from headache, feverishness, chills, muscular pain, joint pain, and fatigue. Said symptom(s) may be noticed by the patient, parent or caregiver. Said at least one respiratory symptom may be at least one symptom selected from coughing, sore throat, and nasal congestion. Preferably, the time point of the onset of influenza symptoms is confirmed by verifying that within 24 hours from the above time point, that the body temperature reaches 38° C. to 40° C. or more.

After administration of the compound to be used in the present invention the plasma concentration of the compound of formula (II) may lead to similar exposures to the ones achieved in non-Asian adult patient population at the dose of 40 mg in the T0831 study, i.e. AUC=3371 ng·h/mL, Cₘₐₓ=56.9 ng/ml and Ca=33.1 ng/mL. Administration of the compound to be used in the present invention preferably leads to an accelerated recovery from the influenza virus infection of the treated patient as compared to an untreated patient to whom the compound has not been administered. Or, in other words, preferably the treated patient to whom the compound to be used in the present invention has been administered has a reduced time to recovery as compared to an untreated patient to whom the compound has not been administered. Herein, the term “untreated patient” means that said patient did not receive the compound to be used in the present invention, i.e. did not receive the compound having the formula (I) or (II) or a pharmaceutically acceptable salt thereof. However, said “untreated patient” may or may not have received another medicament, e.g. another antiviral drug. For example, in the present invention the untreated patient may have been administered with oseltamivir. In one example a patient which receives the compound to be used in the present invention is 1 year or older but younger than 12 years and the untreated patient has been administered with oseltamivir. The treatment regimen of oseltamivir is commonly known in the art. For example, oseltamivir may be administered twice daily for 5 days. Appropriate doses for oseltamivir are based on body weight and commonly known in the art. It is preferred in the present invention that the compound to be used herein leads to a better therapeutic effect as compared to oseltamivir administration.

The treated patient to whom the compound has been administered preferably has a decreased virological activity as compared to an untreated patient to whom the compound has not been administered. For example, it is preferred that the change from baseline in the virus titer is at least −4.20 log₁₀ (TCID₅₀/mL), and/or that the change from baseline in the amount of viral RNA is at least −1.75 log₁₀ (virus particles/mL) on Day 2 (i.e. two days after administration of the compound to be used herein, which is administered on Day 0).

For example, the virological activity may be decreased in the treated patient within 86 hours after administration of the compound to be used in the present invention, and may remain decreased for at least 21.5 hours. Measurement of the virological activity is commonly known in the arg. For example, the virological activity may be measured by:
(a) determination of the time to cessation of viral shedding;
(b) determination of the influenza virus titer; and/or
(c) determination the amount of virus RNA.

In this regard, the duration of influenza virus shedding may be measured as time to shedding cassation following symptom onset. The amount of virus RNA may be measured by using reverse transcriptase-polymerase chain reaction (RT-PCR). The virus titer may be measured in the following manner.
(1) MDCK-SIAT1 cells seeded in a flat-bottom 96-well microplate are cultured in a 5% CO₂ incubator at 37±1° C. for 1 day.
(2) A standard strain (e.g. influenza virus AH3N2, A/Victoria/361/2011, storage condition: −80° C., origin: National Institute of Infectious Diseases), a sample (collected from a patient and stored in an ultra-low-temperature freezer), and a medium for cell control are diluted 101 to 107 folds by a 10-fold serial dilution method.
(3) After cells present in a sheet form are confirmed under an inverted microscope, the medium is removed, and a new medium is added at 100 μL/well.
(4) The medium is removed.
(5) Each of the samples (101 to 107) prepared in (2) above is inoculated at 100 μL/well, using 4 wells per sample.
(6) Centrifugal adsorption is performed at room temperature at 1000 rpm for 30 minutes. (7) After centrifugation, the medium is removed, and cells are washed once with a new medium.
(8) A new medium is added at 100 μL/well.
(9) Incubation is performed in a 5% CO₂ incubator at 33±1° C. for 3 days.
(10) After incubation, the CytoPathic Effect (CPE) is evaluated under an inverted microscope.

It is preferred that the compound to be used in the present invention reduces the time to alleviation of influenza signs and symptoms (TASS) by at least 6 hours, preferably by at least about 12 hours (e.g. by about 24 hours or more) as compared to an untreated patient to whom the compound has not been administered. More specifically, the compound preferably reduces TASS by at least 6 hours, preferably by at least about 12 hours (e.g. by about 24 hours or more) relative to their respective placebos (or relative to an untreated patient). In line with this, it is preferred that the time from diagnosis of the influenza virus infection until recovery is decreased in the treated patient to whom the compound has been administered as compared to an untreated patient to whom the compound has not been administered. In this regard the patient may be classified as being recovered when at least one of the following recovery criteria is met and remains met for at least 21.5 hours:
(a) return to afebrile state (tympanic temperature s 37.2° C.);
(b) a score of 0 (no problem) or 1 (minor problem) for cough and nasal symptoms as specified in items 14 and 15 of the Canadian Acute Respiratory Illness and Flu Scale (CARIFS), preferably a score of 0 (no problem) or 1 (minor problem) for all 18 symptoms specified in the (CARIFS);
(c) cessation of viral shedding; and/or
(d) return to normal health and activity.

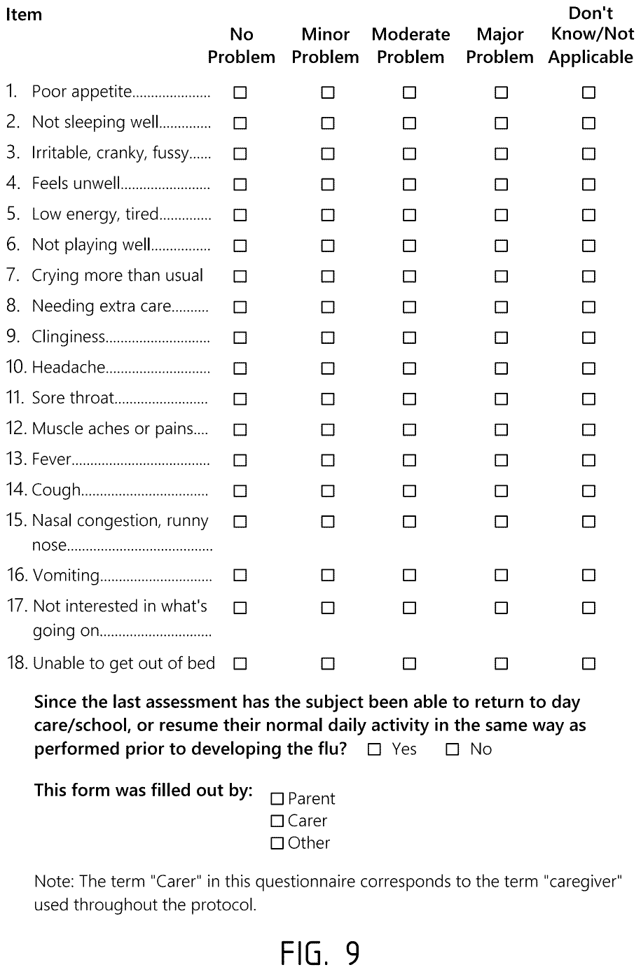

The Canadian Acute Respiratory Illness and Flu Scale (CARIFS) can be used to identify a treatment benefit of the compound to be used in the present invention (e.g. baloxavir marboxil). The CARIFS is commonly known in the art and shown in FIG. 9. The CARIFS is a reliable questionnaire which is composed of 18 questions, each with a 4-point Likert response. The CARIFS questionnaire can be completed by the patient, parent, caregiver and/or physician and covers three domains: symptoms (e.g., cough), function (e.g., play), and parental impact (e.g., clinginess). The CARIFS is calculated as the sum of the items and measures duration of illness.

The return to normal health and activity may be achieved if the patient is able to return to day care or school, and/or to resume his or her normal daily activity in the same way as performed prior to developing the influenza virus infection.

Administration of the compound to be used in the present invention may prevent the occurrence of an influenza-related complication. Said influenza-related complication may be at least one of the complications selected from the group consisting of radiologically confirmed pneumonia, bronchitis, sinusitis, otitis media, encephalitis/encephalopathy, febrile seizures, and myositis. Generally subsequent or partially overlapping with the initial acute viral illness, the most common complications of influenza in children are otitis media, pneumonia (primary influenza virus and secondary bacterial pneumonia), respiratory failure, and seizures (Mistry, Pediatrics 134.3 (2014): e684-e690). These most common complications are preferably prevented in the patient who is treated with the compound to be used in the present invention. It is further envisaged that death of the patient caused by the influenza virus infection is prevented by the administration of the compound. Usually, influenza infected persons do not die from the influenza infection per se but because of the development of a bacterial superinfection. Herein the term “death (of the patient) caused by the influenza virus infection” also includes death which is caused by a bacterial superinfection which had developed in an influenza infected person.

In the context of the present invention it is further envisaged that the requirement of antibiotics is prevented by the administration of the compound. Usually, a bacterial superinfection leads to the requirement of antibiotics. Thus, in accordance with the present invention, a bacterial superinfection may be prevented in the treated patient. Another condition which usually leads to a requirement of antibiotics is an asthma attack. Administration of the compound to be used in the present invention may also prevent hospitalization of the treated patient.

As detailed herein above and below, the compound to be used in the present invention may have the formula (I), (II) or may be a pharmaceutically acceptable salt of the compound of formula (I) or (II). In a preferred aspect of the present invention the compound has the formula (I). The compound to be used in accordance with the present invention may be combined with other anti-influenza drugs. Four antiviral drugs are currently approved in the EU for the prevention and treatment of influenza: the M2 ion-channel inhibitor amantadine and the NAIs oseltamivir phosphate, zanamivir and peramivir. A second M2 inhibitor, rimantadine, holds marketing authorisations in the Czech Republic, France and Poland but is not marketed in these countries. Therefore, the compound to be used in the present invention may be administered as co-therapy with amantadine, oseltamivir phosphate, zanamivir, peramivir, and/or rimantadine. Neuraminidase inhibitors (NAIs) are the mainstay of treatment for influenza infections. Therefore, if the compound to be used in the present invention is administered as co-therapy, then it is preferably combined with oseltamivir phosphate or zanamivir. Both oseltamivir phosphate and zanamivir are administered twice daily for 5 days.

The patient to be treated in the present invention is preferably healthy beside the influenza virus infection. It is preferred that the patient is not treated with any medicament beside the compound to be used in the present invention. For example, it is preferred that the patient is not treated with an investigational therapy, a systemic antiviral drug (e.g. peramivir, Ianinamivir, oseltamivir, zanamivir, rimantadine, umifenovir or amantadine), immunosuppressants, corticosteroids, antifungal drugs, or a drug which is administered to the eyes, nose or ears, or by inhalation. However, if influenza symptoms, such as fever and headache, are so severe (e.g. in the opinion of the patient and/or caregiver) that the patient needs pain treatment, then the compound to be used in context of the present invention may be combined with acetaminophen (i.e. paracetamol). Acetaminophen may be administered at a dose appropriate to the age and body weight of the pediatric patient.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease (e.g., preventing or delaying the worsening of the disease), preventing or delaying the spread (e.g., infection, transmission, etc.) or the disease, delay or slowing the progression of the disease, ameliorating the disease state, providing a remission (whether partial or total) of the disease, decreasing the dose of one or more medications required to treat the disease, enhancing effect of another medication, delaying the progression of the disease, increasing the quality of life, and/or prolonging survival. Also encompassed by “treatment” is a reduction of pathological consequence of influenza. The methods of the invention contemplate any one or more of these aspects of treatment.

The term “effective amount,” as used herein, refers to a sufficient amount of a compound disclosed herein being administered which will relieve to some extent one or more of the symptoms of the disease or condition being treated, e.g., influenza or influenza-related complications. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some examples, an “effective amount” for therapeutic uses is the amount of the composition comprising a compound disclosed herein required to provide a clinically significant increase in disease symptoms or prevent a clinically significant showing in disease symptoms. In some embodiments, an appropriate “effective” amount in any individual case is determined using techniques, such as a dose escalation study.

In one aspect of the present invention the patient does not meet one of the following exclusion criteria:
(i) requires hospitalization (e.g. because of severe symptoms of influenza, complications of influenza or significant comorbidities);
(ii) has concurrent infections requiring systemic antiviral therapy;
(iii) is a preterm neonate (born at <37 weeks gestation) and/or weighing <2.5 kg at screening;
(iv) is obtaining concomitant treatment with steroids or other immunosuppressant therapy;
(v) has an HIV infection or another immunosuppressive disorder;
(vi) has an uncontrolled renal, vascular, neurologic, or metabolic disease (e.g., diabetes, thyroid disorders, adrenal disease), hepatitis, cirrhosis, or pulmonary disease or patients with known chronic renal failure;
(vii) has active cancer at any site;
(viii) has a history of organ transplantation;
(ix) has a known allergy to the compound of the invention or to acetaminophen (also known as paracetamol); and
(x) is a female who has commenced menarche (i.e., child-bearing potential).

The meaning of the term “influenza virus infection” or variations thereof (e.g., “influenza”) is commonly known in the art and refers to a disease which is caused by the influenza virus. More specifically, an influenza virus infection is an acute respiratory infectious disease caused by a virus of the orthomyxovirus family. Two forms are known to principally infect humans and to cause disease in humans, the influenza A virus and the influenza B virus. The influenza viruses have a segmented, negative-sense, single-stranded, lipid encapsulated ribonucleic acid (RNA) genome; they range between 80 and 100 nm in size. Subtypes are defined according to haemagglutinin (HA) and neuraminidase (NA) glycoproteins present in the viral lipid coat. Influenza viruses enter the respiratory epithelial cell by attachment of the viral HA to sialic acid-containing receptors on the cell membrane, followed by internalisation of the virus into an acidic endosome. In the acidic environment of the endosome, the HA undergoes a conformational change that liberates a fusion peptide and results in fusion of the viral envelope with the endosomal membrane. At the same time the matrix-2 (M2) protein acts as an ion channel allowing hydrogen ions to enter the virion from the endosome. This allows the viral gene segments to leave the virion and enter the cytoplasm, a process known as uncoating. Viral gene segments are transported to the nucleus where the viral polymerase complex, composed of the proteins polymerase basic protein 1 (PB1), polymerase basic protein 2 (PB2), and polymerase acidic protein (PA), directs the synthesis of the plus-sense messenger RNA (mRNA) as well as, via a plus-sense full length complementary RNA, synthesis of negative-sense full length copies that will serve as progeny genomic RNA. The polymerase proteins also play a role in disruption of host cell protein synthesis. Assembly of progeny virions occurs at the plasma membrane, and the viral NA protein plays a role in release of virus from the cell surface by cleavage of surface sialic acid.

The “compound” to be used in the present invention is a compound which has one of the following formulae (I) and (II):

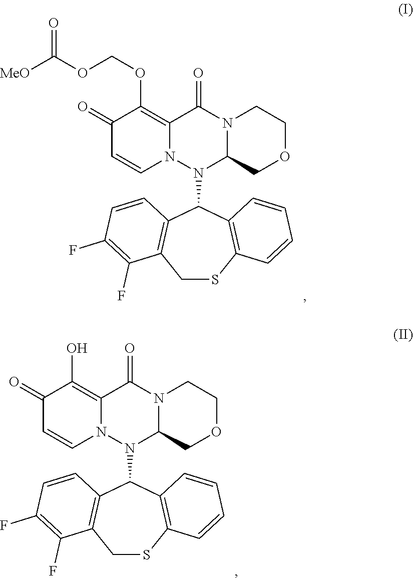 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically acceptable salt (i.e. of the compound having a formula of (I) or (II)). The compound to be used in the present invention is also referred to herein as “compound”, “compound for use”, “compound to be used (herein/in the present invention)” or “compound of the present invention”.

The compound to be used in the present invention acts as a selective cap-dependent endonuclease (CEN) inhibitor, inhibiting the ‘cap-snatching’ function of the PA subunit of the influenza polymerase, which is used to cleave 5′ cap structures from host cell mRNAs, which are used as primers for viral mRNA transcription. By inhibiting this essential function, the compound as used herein suppresses the replication of influenza viruses.

The compound to be used in the present invention has a broad spectrum of activity against seasonal (e.g. A/H1N1, A/H3N2, and B) and highly pathogenic avian (e.g. A/H5N1, A/H7N9) influenza viruses, with more potent antiviral activity (lower half maximal inhibitory concentration [IC₅₀]) compared with other common anti-influenza drugs such as oseltamivir, zanamivir, or peramivir. The compound's ability to be efficacious as a single dose administration simplifies treatment and improves patient compliance compared to neuraminidase inhibitors (NAIs). Preferably, the compound has the formula of (I) or (II), most preferably of (I). The compound of formula (I) can also be displayed as follows:

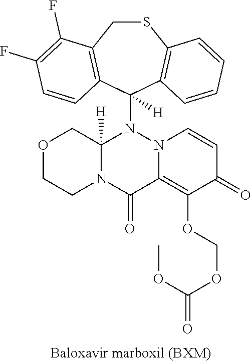 [SMILES: COC(=O)OCOc1c2n(ccc1=O)N([C@@H]1c3ccccc3SCc3c1ccc(F)c3F)[C@@H]1COCCN1C2=O]

This compound (i.e. the compound of formula (I)) has a molecular formula of C₂₇H₂₃F₂N₃O₇S. This compound is a pro-drug which is known as baloxavir marboxil. Baloxavir marboxil is known in the art and described, e.g., in Noshi, Antiviral research 160 (2018): 109-117.

Baloxavir marboxil (i.e. the compound of formula (I)) is an anti-influenza virus drug with a novel mechanism of action. It was discovered and is being developed by Shionogi & Co., Ltd. and F. Hoffman-La Roche, Ltd. Baloxavir marboxil (S-033188) is a pro-drug and is converted to an active form baloxavir (S-033447) through metabolism (hydrolysis). The active form is shown herein as formula (II). The active form baloxavir (S-033447) selectively inhibits cap-dependent endonuclease (CEN) activity necessary for replication of influenza viruses (Omoto, Sci Rep. 2018; 8(1):9633). A broad spectrum of activity against seasonal influenza viruses and on alleviating effects of influenza symptoms were shown in nonclinical efficacy studies and clinical studies in patients with influenza, including the phase 2 proof of concept and dose-finding study, the phase 3 double-blind study in otherwise healthy patients (Portsmouth S, Kawaguchi K, Arai M, Tsuchiya K, Uehara T. Cap-dependent endonuclease inhibitor baloxavir marboxil (S-033188) for the treatment of influenza: results from a phase 3, randomized, double-blind, placebo- and active-controlled study in otherwise healthy adolescents and adults with seasonal influenza. Abstract LB-2. Oral presentation at ID Week 2017, October 4-8 2017, San Diego, CA, USA.), and the Phase 3 open-label study in otherwise healthy pediatric patients.

The compound as shown in formula (II) is the active form of baloxavir marboxil (i.e. of the pro-drug of formula (I)). The compound of formula (I) can also be displayed as follows:

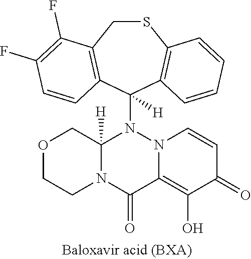 [SMILES: O=C1c2c(O)c(=O)ccn2N([C@@H]2c3ccccc3SCc3c2ccc(F)c3F)[C@@H]2COCCN12]

The compound of formula (II) is also known as baloxavir or baloxavir acid. Baloxavir acid is known in the art and described, e.g., in Noshi, Antiviral research 160 (2018): 109-117.

The pharmaceutically acceptable salts of the compounds used in the present invention include, for example, salts with alkaline metal (e.g., lithium, sodium, potassium or the like), alkaline earth metal (e.g., calcium, barium or the like), magnesium, transition metal (e.g., zinc, iron or the like), ammonia, organic bases (e.g., trimethylamine, triethylamine, dicyclohexylamine, ethanolamine, diethanolamine, triethanolamine, meglumine, ethylenediamine, pyridine, picoline, quinoline or the like) or amino acids, or salts with inorganic acids (e.g., hydrochloric acid, sulfuric acid, nitric acid, carbonic acid, hydrobromic acid, phosphoric acid, hydroiodic acid or the like) or organic acids (e.g., formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid, ethanesulfonic acid or the like). Especially, salts with sodium, potassium, calcium, magnesium, iron and the like are included. These salts can be formed by the usual methods.

The production of the compound of the present invention is well known in the art. For example, the compound of the present invention can be prepared with the methods described in the patent application PCT/JP2016/063139, which is published as WO 2016/175224A1.

As mentioned above, in accordance with the present invention, it is preferred that the influenza virus strain does not comprise a PA protein having a sequence which has at least 80%, preferably at least 90%, more preferably at least 95%, at least 96%, at least 97%, at least 98%, or at least 99% sequence identity to the sequence of SEQ ID NO:1 and comprising a substitution (e.g. an I to T substitution) at the position corresponding to position 138 of SEQ ID NO:1. In particular, FASTA sequences of two sequences of viral PA proteins can be generated and aligned in order to evaluate the degree of identity between the two viral PA proteins. To determine the percent identity of two sequences, the sequences are aligned for optimal comparison purposes (e.g., gaps can be introduced in one or both of a first and a second amino acid sequence for optimal alignment and non-homologous sequences can be disregarded for comparison purposes). The percent identity between the two sequences is a function of the number of identical positions shared by the sequences, taking into account the number of gaps, and the length of each gap, which need to be introduced for optimal alignment of the two sequences. Percent identity between two polypeptides/amino acid sequences is determined in various ways which are known by the skilled person, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482 489 (1981)) as incorporated into GeneMatcher PIus™, Schwarz and Dayhof (1979), Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. Preferably, the viral PA protein sequences are compared over their entire lengths. For purposes of the present invention, the comparison of sequences and determination of percent identity between two sequences can be accomplished using a Blossum 62 scoring matrix (with a gap penalty of 12, a gap extend penalty of 4, and a frameshift gap penalty of 5).

As described above, the present invention provides means and methods for treating an influenza virus infection of patients that are younger than 12 years, in particular by providing an optimized dosage for these pediatric patients. In line with this, the invention also relates to the following aspects. All explanations, definitions and preferred aspects which are explained above and below also relate, mutatis mutandis, to the inventive aspects described below.

The invention also relates to a compound for use in treating an influenza virus infection, wherein the compound has one of the formulae (I) and (II) or its pharmaceutically acceptable salt, and wherein the following dosage is used:
(i) in a patient that is younger than 1 year:
   (a) if the patient is younger than 4 weeks, then the effective amount is 0.8-1.2 mg/kg body weight, preferably about 1 mg/kg body weight;
   (b) if the patient is 4 weeks or older but younger than 3 months, then the effective amount is 0.8-1.2 mg/kg body weight, preferably about 1 mg/kg body weight;
   (c) if the patient is 3 months or older but younger than 12 months, then the effective amount is 1.8-2.2 mg/kg body weight, preferably about 2 mg/kg body weight;
(ii) in a patient that is 1 year or older but younger than 12 years:
   (a) if the patient has a body weight of less than 20 kg, then the effective amount is 1.8-2.2 mg/kg body weight, preferably about 2 mg/kg body weight; or
   (b) if the patient has a body weight of 20 kg or more, then the effective amount is 35-45 mg, preferably about 40 mg.

The invention further relates to a pharmaceutical composition for use in treating an influenza virus infection, wherein the pharmaceutical composition comprises the compound having one of the formulae (I) and (II) or its pharmaceutically acceptable salt, and optionally comprising a pharmaceutically acceptable carrier, wherein the following dosage is used:
(i) in a patient that is younger than 1 year:
   (a) if the patient is younger than 4 weeks, then the effective amount is 0.8-1.2 mg/kg body weight, preferably about 1 mg/kg body weight;
   (b) if the patient is 4 weeks or older but younger than 3 months, then the effective amount is 0.8-1.2 mg/kg body weight, preferably about 1 mg/kg body weight;
   (c) if the patient is 3 months or older but younger than 12 months, then the effective amount is 1.8-2.2 mg/kg body weight, preferably about 2 mg/kg body weight;
(ii) in a patient that is 1 year or older but younger than 12 years:
   (a) if the patient has a body weight of less than 20 kg, then the effective amount is 1.8-2.2 mg/kg body weight, preferably about 2 mg/kg body weight; or
   (b) if the patient has a body weight of 20 kg or more, then the effective amount is 35-45 mg, preferably about 40 mg.

Also encompassed by the present invention is a method for treating influenza, comprising: reading a dosage instruction on a package insert or in a package for a pharmaceutical formulation comprising a compound having one of the formulae (I) and (II) or being a pharmaceutically salt thereof; and administering an effective amount of the compound to an influenza-infected patient, and wherein the following dosage is used:
(i) in a patient that is younger than 1 year:
   (a) if the patient is younger than 4 weeks, then the effective amount is 0.8-1.2 mg/kg body weight, preferably about 1 mg/kg body weight;
   (b) if the patient is 4 weeks or older but younger than 3 months, then the effective amount is 0.8-1.2 mg/kg body weight, preferably about 1 mg/kg body weight;
   (c) if the patient is 3 months or older but younger than 12 months, then the effective amount is 1.8-2.2 mg/kg body weight, preferably about 2 mg/kg body weight;
(ii) in a patient that is 1 year or older but younger than 12 years:
   (a) if the patient has a body weight of less than 20 kg, then the effective amount is 1.8-2.2 mg/kg body weight, preferably about 2 mg/kg body weight; or
   (b) if the patient has a body weight of 20 kg or more, then the effective amount is 35-45 mg, preferably about 40 mg.

The invention also relates the use of a compound which has one of the formulae (I) and (II), or its pharmaceutically acceptable salt, for the preparation of a medicament for treating an influenza-infected patient, wherein the following dosage is used:
(i) in a patient that is younger than 1 year:
   (a) if the patient is younger than 4 weeks, then the effective amount is 0.8-1.2 mg/kg body weight, preferably about 1 mg/kg body weight;
   (b) if the patient is 4 weeks or older but younger than 3 months, then the effective amount is 0.8-1.2 mg/kg body weight, preferably about 1 mg/kg body weight;
   (c) if the patient is 3 months or older but younger than 12 months, then the effective amount is 1.8-2.2 mg/kg body weight, preferably about 2 mg/kg body weight;
(ii) in a patient that is 1 year or older but younger than 12 years:
   (a) if the patient has a body weight of less than 20 kg, then the effective amount is 1.8-2.2 mg/kg body weight, preferably about 2 mg/kg body weight; or
   (b) if the patient has a body weight of 20 kg or more, then the effective amount is 35-45 mg, preferably about 40 mg.

Also provided by the present invention is a package comprising a pharmaceutical formulation comprising a compound which has one of the formulae (I) and (II), or its a pharmaceutically salt, and further comprising a dosage instruction for administering an effective amount of the compound to an influenza-infected patient, wherein the following dosage is used:
(i) in a patient that is younger than 1 year:
   (a) if the patient is younger than 4 weeks, then the effective amount is 0.8-1.2 mg/kg body weight, preferably about 1 mg/kg body weight;
   (b) if the patient is 4 weeks or older but younger than 3 months, then the effective amount is 0.8-1.2 mg/kg body weight, preferably about 1 mg/kg body weight;
   (c) if the patient is 3 months or older but younger than 12 months, then the effective amount is 1.8-2.2 mg/kg body weight, preferably about 2 mg/kg body weight;
(ii) in a patient that is 1 year or older but younger than 12 years:
   (a) if the patient has a body weight of less than 20 kg, then the effective amount is 1.8-2.2 mg/kg body weight, preferably about 2 mg/kg body weight; or
   (b) if the patient has a body weight of 20 kg or more, then the effective amount is 35-45 mg, preferably about 40 mg.

As mentioned above, one aspect of the present invention relates to a pharmaceutical composition comprising a compound which has one of the formulae (I) and (II), or its pharmaceutically acceptable salt, and optionally comprising a pharmaceutically acceptable carrier. The pharmaceutical compositions can be formulated with a pharmaceutically acceptable carrier by known methods. For example, the compositions can be formulated by appropriately combining the ingredients with a pharmaceutically acceptable carrier or a medium, specifically, sterile water or physiological saline, vegetable oils, emulsifiers, suspending agents, surfactants, stabilizers, flavoring agents, excipients, vehicles, preservatives, binding agents, and such, by mixing them at a unit dose and form required by generally accepted pharmaceutical implementations. Specific examples of the carriers include light anhydrous silicic acid, lactose, crystalline cellulose, mannitol, starch, carmellose calcium, carmellose sodium, hydroxypropyl cellulose, hydroxypropyl methylcellulose, polyvinylacetal diethylaminoacetate, polyvinylpyrrolidone, gelatin, medium-chain triglyceride, polyoxyethylene hardened castor oil 60, saccharose, carboxymethyl cellulose, corn starch, inorganic salt, and such. The content of the active ingredient in such a formulation is adjusted so that an appropriate dose within the required range can be obtained.

The pharmaceutical composition may optionally comprise one or more pharmaceutically acceptable excipients, such as carriers, diluents, fillers, disintegrants, lubricating agents, binders, colorants, pigments, stabilizers, preservatives, antioxidants, or solubility enhancers. Also, the pharmaceutical compositions may comprise one or more solubility enhancers, such as, e.g., poly(ethylene glycol), including poly(ethylene glycol) having a molecular weight in the range of about 200 to about 5,000 Da, ethylene glycol, propylene glycol, non-ionic surfactants, tyloxapol, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, glucosyl-α -cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate, or any combination thereof.

The pharmaceutical compositions are not limited to the means and methods described herein. The skilled person can use his/her knowledge available in the art in order to construct a suitable composition. Specifically, the pharmaceutical compositions can be formulated by techniques known to the person skilled in the art such as the techniques published in Remington's Pharmaceutical Sciences, 20^{th} Edition.

### EXAMPLES

The Examples illustrate the invention. The invention will be more fully understood by reference to the examples described herein. The claims should not, however, be construed as limited to the scope of the examples.

### Example 1: Materials and Methods of the Simulation of Pediatric Doses

### 1. Population Pharmacokinetic (PK) Analysis

Population PK analysis were conducted using Japanese pediatric patient study information.

### 1.1 Background Data

Following background data available for subjects were summarized and were used as the candidate of covariates: age (years and weeks), body weight, body mass index (BMI), aspartate aminotransferase (AST), alanine aminotransferase (ALT), total bilirubin (Tbil), estimated glomerular filtration rate (eGFR), and creatinine clearance (CLcr) at baseline as continuous data, and gender (male, female), race (“Asian”, “Non-Asian”, wherein the “Non-Asian” group reflects, e.g., white such as Caucasian patients), health status (otherwise healthy patients with influenza, or patients without influenza) and food conditions (dosing ≥4 hours before and ≥4 hours after food intake [fasted], dosing within 2 to 4 hours before or 2 to 4 hours after food intake [intermediate], or dosing <2 hours before or <2 hours after food intake [fed]) as categorical data. Background data at baseline were obtained from observations prior to or on the first day of dosing or at screening if this value was not available. The eGFR was calculated by Schwartz formula (Schwartz, Pediatric Clinics of North America. 1987; 34: 571-90). CLcr for pediatrics was calculated from eGFR and body surface area (BSA). The BSA was calculated using the following equation reported by Mostellar (Mosteller, N Eng J Med. 1987; 317:1098).

$${BSA ( {m}^{2} ) = [ height ( cm ) \times body weight ( kg ) / 3600 ]}^{1 / 2}$$

The following equations were used to calculate eGFR and CLcr.

| Parameter | Age | Equation |
|---|---|---|
| eGFR | 2 to 11 years | eGFR = 0.55 × [height (cm)]/Scr |
| (mL/min/1.73 m²) | | |
| | Birth to 1 year | eGFR = 0.45 × [height (cm)]/Scr |
| | (Full-term | |
| | infants) | |
| CLcr (mL/min) | <12 years | CLcr = eGFR × BSA/1.73 |

BSA = body surface area (m²);
Ser = serum creatinine (mg/dL)

### 1.2 Base Model

A 2-compartment model with first-order absorption and lag time was initially tested for describing plasma concentration of baloxavir (S-033447), because it is the same structural model that was previously selected to describe the data in pediatric patients (Ishibashi T.

Population Pharmacokinetics of S-033188 (Pediatric Patient). Study Report (Final, Study No.: S-033188-CB-273-N). Shionogi & Co., Ltd.; 2017). The 2-compartment model includes the following parameters: apparent total clearance (CL/F), apparent volume of central and peripheral compartments (Vc/F and Vp/F), apparent inter-compartmental clearance (Q/F), first-order rate of absorption (Ka), and absorption lag time (ALAG). The difference of systemic exposure among formulations was incorporated in the model as the difference of relative bioavailability (F). F is 1 for to-be-marketed 20-mg tablet and 0.88 for to-be-marketed 10-mg tablet (A Phase 1 Study to Evaluate the Bioequivalence of S-033188 10-mg and 20-mg Tablets and Effect of Food on the Pharmacokinetics in Healthy Adults. Clinical Study Report (Study No. 1622T081F). Shionogi & Co., Ltd.; 2017). F was set to 1 for 2% granule in this study because 2% granule and 20-mg tablet is bioequivalent (Study No. 1703T081G) (A Phase 1 Study to Evaluate the Bioequivalence of S-033188 20-mg Tablet and S-033188 Granules 2%. Clinical Study Report (Study No. 1703T081G). Shionogi & Co., Ltd.; 2018).

Individual model parameters were estimated based on a fixed effect parameter (PKP) and an inter-individual variability (IIV) for certain PK parameters which are assumed to follow a log-normal distribution and exponential error model as described in Equation (1):

$${PKP}_{i} = PKP \times exp ( {η}_{PKP , i} ) \qquad ( 1 )$$

where PKPᵢ represents the i-th individual value of PK parameters, PKP represents the typical value of population PK parameters, and η_{PKP,i} denotes the difference between the i-th individual and typical PK parameter. The η_{PKP} is a random variable of the IIV parameters and normally distributed with a mean of 0 and a variance of ω_{PKP}².

After model building, the covariance between pairs of random IIV parameters were examined graphically by plotting η_{PKP,i} in different PK parameters and covariance might be added as appropriate to account for observed correlations. Decisions regarding the inclusion of covariance of IIV were based on the numerical stability of the resulting model or on the goodness-of-fit (GOF) plots as described in Section 1.5.

Shrinkage in each η_{PKP} (sh_η_{PKP}) was computed in NONMEM.

The additive error model, the proportional error model and/or the combination error model (the additive error+the proportional error model) were tested as an intra-individual (residual) variability. The additive error model, the proportional error model and the combination error model are given in the following equations.

$${C}_{ij} = {C}_{ij} ( pred ) + {ε}_{1 , ij} : additive error model \qquad ( 2 )$$

$${C}_{ij} = {C}_{ij} ( pred ) + ( 1 + {ε}_{1 , ij} ) : proportional error model \qquad ( 3 )$$

$${C}_{ij} = {C}_{ij} ( pred ) + ( 1 + {ε}_{1 , ij} ) + {ε}_{2 , ij} : combination error model \qquad ( 4 )$$

where Cᵢⱼ represents the observed j-th concentration in the i-th individual, Cᵢⱼ (pred) represents the j-th concentration predicted from the i-th individual PK parameters and ε (ε_{1,ij}, ε_{2,ij}) denotes the difference between the j-th observed and predicted concentration in the i-th individual. The ε (ε_{1,ij}, ε_{2,ij}) is a random variable of the intra-individual variability parameters from population mean and normally distributed with a mean of 0 and a variance of σ² (σ₁², σ₂²).

Shrinkage in ε (sh_ε) was computed in NONMEM.

Error model for intra-individual variability was selected by the diagnostic plots described in Section 1.5 and/or the value of objective function value (OBJ) at the statistical significance level of 0.05 (p<0.05) based on χ² test, that is, difference in OBJ (ΔOBJ) of less than −3.84 for one degree of freedom represents a statistically significant model improvement.

The structure of the base model with error models was expanded as necessary to best reflect the characteristic shape of the observations over time. When IIV could not be estimated appropriately, removal of its IIV term was considered.

### 1.3 Covariate Model

After building a base model with selection of an error model for intra-individual variability, the influence of background data was assessed to build a covariate model. Covariate model was constructed by means of combination of screening for covariates, forward selection, and stepwise backward deletion. The significance level of 0.05 based on χ² test (p<0.05) was used for the screening (ΔOBJ was less than −3.84 for one degree of freedom). The significant covariates at screening were tested in the forward selection at the significance level of 0.05 based on λ² test to construct a full model (ΔOBJ was less than −3.84 for one degree of freedom). The significance level of 0.01 based on λ² test was used for the stepwise backward deletion to construct a final model (ΔOBJ was more than 6.63 for one degree of freedom).

As the first covariate assessment, body weight was tested on CL/F and Vc/F because body weight is considered to be the most significant covariate in pediatrics. Body weight was tested as a covariate on the other PK parameters (e.g., Vp/F, Q/F etc.).

For body weight, a power model as shown in Equation (5) was used.

$$PKP = {θ}_{1} \times {( COV / median of COV )}^{θ 2} \qquad ( 5 )$$

where COV is a values of the covariate and θ₁, θ₂ are the typical values of model parameters to be estimated in equation. The typical allometric exponents of 0.75 on CL/F and Q/F, and 1 on Vc/F and Vp/F (Holford, Clin. Pharmacokinet. 1996; 30: 329-32; Anderson, Annu Rev Pharmacol Toxicol. 2008; 48: 303-32) were tested for e₂ for the effect of body weight on clearance and volume of distribution. Also, exponents of 0.632 on CL/F and Q/F, and 1.03 on Vc/F and Vp/F, which were estimated in the previous pediatric population PK model for baloxavir (S-033447) (Ishibashi T. Population Pharmacokinetics of S-033188 (Pediatric Patient). Study Report (Final, Study No.: S-033188-CB-273-N). Shionogi & Co., Ltd.; 2017; published (Koshimichi, Journal of Pharmaceutical Sciences (2019) 1-6, https://doi.org/10.1016/j.xphs.2019.04.010), were tested for e₂ for the effect of body weight on clearance and volume of distribution.

In addition to body weight, age (weeks), BMI, gender, AST, ALT, Tbil, eGFR, CLcr, and health status were tested as a covariate on CL/F; age (weeks), BMI, gender, and health status were tested as a covariate on Vc/F; age (weeks), gender, health status and food conditions were tested as a covariate on Ka; and food conditions was tested as a covariate on F. Background data was tested as a covariate on the other PK parameters (e.g., Vp/F, Q/F etc.).

Prior to building covariate models, plots for relationships between covariates and PK parameters were generated for visual inspection of covariates based on the base model.

For continuous covariates, a power model as shown in Equation (6) was used.

$$PKP = {θ}_{1} \times {( COV / median of COV )}^{θ 2} \qquad ( 6 )$$

where COV is a values of the covariate and θ₁, θ₂ are the typical values of model parameters to be estimated in equation.

For binary and categorical covariates, a multiplicative model as shown in Equation (7) was used.

$$PKP = {θ}_{CAT = 0} \times {( {θ}_{CAT \_ i} )}^{CAT \_ i} \qquad ( 7 )$$

where CAT_i is a series of indicator variables with a value of either 0 or 1 assigned (CAT_1, CAT_2, . . . , CAT_n representing the n levels of CAT; e.g., CAT_1=0 for male and CAT_1=1 for female), and θ_{CAT=0} is the typical values of model parameters to be estimated when the individual categorical covariate index variable is equal to zero and θ_{CAT_i} is the i-th relative influence of model parameters to be estimated for categorical covariate index variable when CAT_i is equal to one.

After building the final model, for a simulation purpose for younger children aged <2 years, a sigmoid hyperbolic model was incorporated in the model (simulation model) to describe the maturation of CL/F. Maturation factor (MF) is described in Equation (8), and CL/F is multiplied by MF.

$$MF = {PMA}^{γ} / ( {PMA}^{γ} + {TM}_{50}^{γ} ) \qquad ( 8 )$$

where PMA is postmenstrual age (weeks), TM₅₀ is maturation half-life (weeks), and γ is hill coefficient. PMA was calculated as 40+age (weeks), assuming that all patients were full-term delivery. The values of TM₅₀ and γ for baloxavir (S-033447) were estimated from data. Also, the values of TM₅₀=54.2 weeks and γ=3.92 for morphine, which is metabolized by uridine diphosphate glucuronosyl transferase (UGT) (Anderson, Paediatr Anaesth. 2011; 21: 222-37), were tested. The model with the smallest OBJ was selected as the simulation model.

Alternative expressions might be considered for continuous covariates based on trends that were observed in covariate plots and alternative expressions might be considered for categorical covariates to facilitate the interpretation of the typical parameter estimates with respect to specific patient categories.

Highly correlated covariates might be tested in separate models in order to avoid confounding in the estimation of covariate effects.

A covariate might be retained in the final model, despite not meeting the criteria above, if there is a strong pharmacological or physiological rationale for its inclusion.

### 1.4 Parameter Estimation

The population PK parameters were estimated for the plasma baloxavir (S-033447) concentration data by NONMEM. The first-order conditional estimation method with interaction (FOCE-1) was used for the analysis.

### 1.5 Model Evaluation

The base and final model were evaluated by using the point estimates of PK parameters and their relative standard error. Also, the following GOF plots with reference lines (identity, zero line, etc.) were generated for model diagnostics.
Observed concentrations (OBS) versus population predicted concentrations (PRED) in both linear and log scale with a line of identity and a trend line
OBS versus Bayesian-predicted individual concentrations (IPRED) in both linear and log scale with a line of identity and a trend line
Conditional weighted residuals (CWRES) or conditional weighted residuals with interaction (CWRESI) versus PRED with a zero line and a trend line
   |Individual weighted residuals (IWRES)| versus IPRED with a trend line
CWRES or CWRESI versus time after reference dose (TARD)
Histogram (optionally QQ plot) of CWRES or CWRESI and WRES
Plots of empirical Bayesian estimate (EBE) of parameters (only base model) and ETAs versus the potential covariates
A scatter plot matrix of EBE of ETAs (only final model)
Distributions (e.g., histograms) of EBE of ETAs (only final model)
OBS, IPRED and PRED concentrations versus time overlaid by individual for representative subjects (secondary any given subjects) (only final model)

The PRED, IPRED, CWRES, CWRESI and WRES are the reserved terms in NONMEM.

The final model should meet the following criteria:
A “minimization successful” statement is indicated by NONMEM.
A covariance step is completed without warning messages by NONMEM.
The number of significant digits is 3 for all estimated 6.
Final estimates of 6 are not close to boundaries.
GOF plots do not indicate unexplained trends.

A final model that did not meet these criteria might be accepted only after careful consideration of the modeling strategy and study objectives.

The predictive performance of a final model was evaluated by prediction-corrected visual predictive check (pcVPC) (Bergstand, AAPS J. 2011; 13: 143-51) and calculating the percentage of the observations outside the 90% prediction intervals (PI). In addition to the pcVPC, the final model was also evaluated by bootstrapping technique (Ette, Journal of clinical pharmacology. 1997, 37 (6): 486-95). At least 200 bootstrap replications were performed and the associated mean parameter estimates and their corresponding 95% confidence interval (CI) were derived from the replicates.

### 1.6 Individual Post-Hoc Pharmacokinetic Parameters

The individual systemic exposures of baloxavir (S-033447), such as Cₘₐₓ, the area under the plasma concentration-time curve from time zero to infinity (AUC_{0-inf}), and C₂₄ after a single dose of baloxavir marboxil (S-033188) were calculated using individual post-hoc PK parameters with empirical Bayesian estimations of the final model. Also, these exposures were calculated using individual post-hoc PK parameters with empirical Bayesian estimations of the simulation model. The formulae needed to calculate the exposure metrics depends on the model structure.

### 1.7 Monte-Carlo Simulation

Monte-Carlo simulation was employed with the final model to assess the relationship between body weight and PK parameters (Cₘₐₓ, AUC_{0-inf}, and C₂₄). A thousand virtual pediatric patients were generated for every 5 kg by simulating the body weight (10 to <60 kg) based on the final model to be assumed as a uniform distribution for body weight.

Also, Monte-Carlo simulation was employed with the simulation model to assess the relationships between age (0 months to <2 years old) and PK parameters (Cₘₐₓ, AUC_{0-inf}, and C₂₄). A thousand virtual pediatric patients were generated for every month old by simulating the age based on the simulation model. The relationship between age and body weight for Japanese pediatrics followed the database by Ministry of Health, Labour and Welfare (Ministry of Health, Labour and Welfare. Research for growth of babies (2010), available at the world-wide-web site e-stat.go.jp/SG1/estat/Xlsdl.do?sinfid=000012673573). To generate virtual pediatric patients, log-normal distribution was assumed for body weight and geometric mean and its coefficient of variance were set for each month (Table 3), and 1:1 proportion was assumed for gender. MF was calculated for each month by equation 8 assuming that all pediatric patients are full-term delivery and their ages are middle in the age range. For example, a pediatric patient with 6 months old, his/her PMA is 40 weeks+6.5 months=68.2 weeks.

TABLE 3
The Relationships between Age and Body Weight for Birth to <2 Years Old Pediatrics

| | Percentile | | | | | | | Assumed | | Maturation |
|---|---|---|---|---|---|---|---|---|---|---|
| Age (months) | 3 | 10 | 25 | 50 | 75 | 90 | 97 | Geometric Mean | CV % | Factor |

(a) Boys

| | | | | | | | | | | |
|---|---|---|---|---|---|---|---|---|---|---|
| 0 to 1 | 2.55 | 2.91 | 3.23 | 3.57 | 3.89 | 4.17 | 4.47 | 3.57 | 17.8 | 0.551 |
| 1 to 2 | 3.53 | 3.94 | 4.35 | 4.79 | 5.22 | 5.59 | 5.96 | 4.79 | 16.3 | 0.626 |
| 2 to 3 | 4.41 | 4.88 | 5.34 | 5.84 | 6.33 | 6.76 | 7.18 | 5.84 | 14.9 | 0.679 |
| 3 to 4 | 5.12 | 5.61 | 6.10 | 6.63 | 7.16 | 7.62 | 8.07 | 6.63 | 13.8 | 0.728 |
| 4 to 5 | 5.67 | 6.17 | 6.67 | 7.22 | 7.76 | 8.25 | 8.72 | 7.22 | 12.8 | 0.770 |
| 5 to 6 | 6.10 | 6.60 | 7.10 | 7.66 | 8.21 | 8.71 | 9.20 | 7.66 | 12.1 | 0.804 |
| 6 to 7 | 6.44 | 6.94 | 7.44 | 8.00 | 8.56 | 9.07 | 9.57 | 8.00 | 11.5 | 0.832 |
| 7 to 8 | 6.73 | 7.21 | 7.71 | 8.27 | 8.84 | 9.36 | 9.87 | 8.27 | 11.0 | 0.856 |
| 8 to 9 | 6.96 | 7.44 | 7.94 | 8.50 | 9.08 | 9.61 | 10.14 | 8.50 | 10.7 | 0.875 |
| 9 to 10 | 7.16 | 7.64 | 8.13 | 8.70 | 9.29 | 9.83 | 10.37 | 8.70 | 10.4 | 0.892 |
| 10 to 11 | 7.34 | 7.81 | 8.31 | 8.88 | 9.48 | 10.03 | 10.59 | 8.88 | 10.1 | 0.905 |
| 11 to 12 | 7.51 | 7.98 | 8.48 | 9.06 | 9.67 | 10.23 | 10.82 | 9.06 | 10.0 | 0.917 |
| 12 to 13 | 7.68 | 8.15 | 8.65 | 9.24 | 9.86 | 10.44 | 11.04 | 9.24 | 9.8 | 0.927 |
| 13 to 14 | 7.85 | 8.32 | 8.83 | 9.42 | 10.05 | 10.65 | 11.28 | 9.42 | 9.7 | 0.935 |
| 14 to 15 | 8.02 | 8.49 | 9.00 | 9.60 | 10.25 | 10.86 | 11.51 | 9.60 | 9.6 | 0.942 |
| 15 to 16 | 8.19 | 8.67 | 9.18 | 9.79 | 10.44 | 11.08 | 11.75 | 9.79 | 9.5 | 0.949 |
| 16 to 17 | 8.36 | 8.84 | 9.35 | 9.97 | 10.64 | 11.29 | 11.98 | 9.97 | 9.4 | 0.954 |
| 17 to 18 | 8.53 | 9.01 | 9.53 | 10.16 | 10.84 | 11.51 | 12.23 | 10.16 | 9.3 | 0.958 |
| 18 to 19 | 8.70 | 9.18 | 9.71 | 10.35 | 11.04 | 11.73 | 12.47 | 10.35 | 9.2 | 0.963 |
| 19 to 20 | 8.86 | 9.35 | 9.89 | 10.53 | 11.25 | 11.95 | 12.71 | 10.53 | 9.2 | 0.966 |
| 20 to 21 | 9.03 | 9.52 | 10.06 | 10.72 | 11.45 | 12.17 | 12.96 | 10.72 | 9.1 | 0.969 |
| 21 to 22 | 9.19 | 9.69 | 10.24 | 10.91 | 11.65 | 12.39 | 13.20 | 10.91 | 9.1 | 0.972 |
| 22 to 23 | 9.36 | 9.86 | 10.41 | 11.09 | 11.85 | 12.61 | 13.45 | 11.09 | 9.1 | 0.974 |
| 23 to 24 | 9.52 | 10.03 | 10.59 | 11.28 | 12.06 | 12.83 | 13.69 | 11.28 | 9.0 | 0.977 |

(b) Girls

| | | | | | | | | | | |
|---|---|---|---|---|---|---|---|---|---|---|
| 0 to 1 | 2.52 | 2.82 | 3.10 | 3.41 | 3.71 | 3.98 | 4.25 | 3.41 | 16.2 | 0.551 |
| 1 to 2 | 3.39 | 3.73 | 4.08 | 4.47 | 4.86 | 5.20 | 5.54 | 4.47 | 14.8 | 0.620 |
| 2 to 3 | 4.19 | 4.58 | 4.97 | 5.42 | 5.86 | 6.27 | 6.67 | 5.42 | 13.7 | 0.679 |
| 3 to 4 | 4.84 | 5.25 | 5.67 | 6.15 | 6.64 | 7.08 | 7.53 | 6.15 | 12.8 | 0.728 |
| 4 to 5 | 5.35 | 5.77 | 6.21 | 6.71 | 7.23 | 7.70 | 8.18 | 6.71 | 12.1 | 0.770 |
| 5 to 6 | 5.74 | 6.17 | 6.62 | 7.14 | 7.67 | 8.17 | 8.67 | 7.14 | 11.6 | 0.804 |
| 6 to 7 | 6.06 | 6.49 | 6.95 | 7.47 | 8.02 | 8.53 | 9.05 | 7.47 | 11.2 | 0.832 |
| 7 to 8 | 6.32 | 6.75 | 7.21 | 7.75 | 8.31 | 8.83 | 9.37 | 7.75 | 10.8 | 0.856 |
| 8 to 9 | 6.53 | 6.97 | 7.43 | 7.97 | 8.54 | 9.08 | 9.63 | 7.97 | 10.6 | 0.875 |
| 9 to 10 | 6.71 | 7.15 | 7.62 | 8.17 | 8.74 | 9.29 | 9.85 | 8.17 | 10.5 | 0.892 |
| 10 to 11 | 6.86 | 7.31 | 7.78 | 8.34 | 8.93 | 9.49 | 10.06 | 8.34 | 10.3 | 0.905 |
| 11 to 12 | 7.02 | 7.46 | 7.95 | 8.51 | 9.11 | 9.68 | 10.27 | 8.51 | 10.3 | 0.917 |
| 12 to 13 | 7.16 | 7.62 | 8.11 | 8.68 | 9.29 | 9.87 | 10.48 | 8.68 | 10.2 | 0.927 |
| 13 to 14 | 7.31 | 7.77 | 8.27 | 8.83 | 9.47 | 10.07 | 10.69 | 8.85 | 10.2 | 0.935 |
| 14 to 15 | 7.46 | 7.93 | 8.43 | 9.03 | 9.56 | 10.27 | 10.90 | 9.03 | 10.1 | 0.942 |
| 15 to 16 | 7.61 | 8.08 | 8.60 | 9.20 | 9.85 | 10.47 | 11.12 | 9.20 | 10.1 | 0.949 |
| 16 to 17 | 7.75 | 8.24 | 8.76 | 9.38 | 10.04 | 10.67 | 11.33 | 9.38 | 10.1 | 0.954 |
| 17 to 18 | 7.90 | 8.39 | 8.93 | 9.55 | 10.23 | 10.87 | 11.55 | 9.55 | 10.1 | 0.958 |
| 18 to 19 | 8.05 | 8.55 | 9.09 | 9.73 | 10.42 | 11.08 | 11.77 | 9.73 | 10.1 | 0.963 |
| 19 to 20 | 8.20 | 8.71 | 9.26 | 9.91 | 10.61 | 11.28 | 11.99 | 9.91 | 10.1 | 0.966 |
| 20 to 21 | 8.34 | 8.86 | 9.43 | 10.09 | 10.81 | 11.49 | 12.21 | 10.09 | 10.1 | 0.969 |
| 21 to 22 | 8.49 | 9.02 | 9.59 | 10.27 | 11.00 | 11.70 | 12.44 | 10.27 | 10.2 | 0.972 |
| 22 to 23 | 8.64 | 9.18 | 9.76 | 10.46 | 11.20 | 11.92 | 12.67 | 10.46 | 10.2 | 0.974 |
| 23 to 24 | 8.78 | 9.34 | 9.93 | 10.64 | 11.40 | 12.13 | 12.90 | 10.64 | 10.2 | 0.977 |

### 2. Software

PK calculations were performed by using WinNonlin (Version 6.2.1). SAS (Version 9.2) was used for statistical analyses. R (Version 3.0.2) was used for PK/PD analysis. NONMEM (Version 7.3), Intel Visual FORTRAN Compiler (version 2010), and Perl-speaks-NONMEM (version 4.2) were used for population PK analysis.

### Example 2: Population Pharmacokinetic Parameters

A population PK model has been developed to describe baloxavir PK in both Japanese and non-Japanese influenza patients (adults and adolescents) who are otherwise healthy (T0821 and T0831). The relationship between drug exposure and various covariates has been explored. The population PK model parameters are summarised in Table 4. Likewise, a paediatric population PK model has been developed to describe the population PK of baloxavir in Japanese otherwise healthy patients aged ≥6 months to <12 years (Study T0822, also called 1618T0822; and Study T0833, also called 1705T0833). The population PK model parameters in paediatrics are summarised in Table 5.

TABLE 4
Population Pharmacokinetic Parameters
in Adults (report S-033188-CB-272-N)

| Pharmacokinetic | | | | |
|---|---|---|---|---|
| parameter | Units | Estimate | RSE (%) | IIV (%) |
| CL/F | L/hr | 5.40 | 1.5 | 38.7 |
| Vc/F | L | 333 | 2.7 | 54.8 |
| Q/F | L/hr | 6.27 | 4.5 | — |
| Vp/F | L | 212 | 2.3 | 22.2 |
| Ka | 1/hr | 1.10 | 6.5 | 111.8 |
| ALAG | hr | 0.32 | 3.6 | — |

CL/F (L/hr) = 5.40 × (body weight/64.8)^{1.04} × 1.72 Non-Asian × (ALT/17)^{-0.115}, where Non-Asian = 1 for Non-Asian and Non-Asian = 0 for Asian
Vc/F (L) = 333 × (body weight/64.8)^{1.76} × 1.36 Non-Asian
Q/F (L/hr) = 6.27 × (body weight/64.8)^{0.473}
Vp/F (L) = 212 × (body weight/64.8)^{0.642}
Ka (hr⁻¹) = 1.10 × 0.613 gender, where gender = 1 for female and gender = 0 for male
Effect of food on bioavailability = 0.869^{fed} where fed = 1 when dosing <2 hours before or after food intake and fed = 0 when dosing ≥2 hours before or after food intake
Abbreviations:
ALAG, absorption lag time;
CL/F, apparent total clearance;
Ka, first-order rate of absorption;
Q/F, apparent inter-compartmental clearance;
Vc/F, apparent volume of central compartment;
Vp/F, apparent volume of peripheral compartment.

TABLE 5
Population Pharmacokinetic Parameters in Japanese paediatrics-
studiesT0822 (1618T0822) and T0833 (1705T0833)

| Pharmacokinetic | | | | |
|---|---|---|---|---|
| parameter | Units | Estimate | RSE (%) | IIV (%) |
| CL/F | L/hr | 2.72 | 5.4 | 22.7 |
| Vc/F | L | 117 | 11.9 | — |
| Q/F | L/hr | 1.06 | 36.7 | — |
| Vp/F | L | 67.1 | 34.4 | — |
| Ka | 1/hr | 0.702 | 17.9 | 128.1 |
| ALAG | hr | 0.47 | 4.1 | |

CL/F (L/hr) = 2.72 × (body weight/20.7)^{0.77}
Vc/F (L) = 117 × (body weight/20.7)^{1.07}
Q/F (L/hr) = 1.06 × (body weight/20.7)^{0.77}
Vp/F (L) = 67.1 × (body weight/20.7)^{1.07}
Relative bioavailability for 10-mg tablet = 0.88 (fixed)
Abbreviations:
ALAG, absorption lag time;
CL/F, apparent total clearance;
Ka, First-order rate of absorption;
Q/F, apparent inter-compartmental clearance;
Vc/F, apparent volume of central compartment;
Vp/F, apparent volume of peripheral compartment.

Baloxavir PK was found to be linear with respect to dose in both adults and paediatrics. PK was found to be well described using a two-compartment model with first-order absorption with a lag-time and first order elimination from the central compartment. In adults, baloxavir demonstrated low oral clearance of 5.4 L/hr (Japanese). Both bodyweight and race (Asian versus non-Asian) were found to be significant covariates on CL/F. At the same bodyweight, CL/F was on average 1.7 fold higher in non-Japanese. Interestingly, a similar but slightly lower ethnic effect was seen on volume (V/F), suggesting the covariate may not solely reflect a difference in absolute bio-availability (F). In Japanese paediatrics, bodyweight was a significant covariate on both clearance and volume. Population median oral clearance was about 3 L/h for a Japanese child weighing 24 kg. Oral drug clearance and inter-compartmental clearance scaled to bodyweight with an allometric exponent of 0.632, whereas both central and peripheral volume terms scaled with their typical exponent approaching 1. Based on these allometric relationships, bodyweight-adjusted oral drug clearance (L/hr/kg) decreases with increasing bodyweight and can be estimated to be about 2-fold lower in a 10-kg child compared to an adult of 70 kg. Furthermore, because volume of distribution scaled roughly proportional to bodyweight (i.e., approximately constant on a per kg basis), disposition half-life increases with increasing bodyweight.

### Example 3: Dose Finding for Non-Asian (e.g. White) Paediatric Patients

A single dose administration will be used, as supported by adult and adolescent phase 2/3 studies as well as phase 3 Japanese paediatric studies, where a single oral dose administration was confirmed to provide rapid and sustained relief of influenza symptoms.

Optimal doses for two non-Asian paediatric patient groups (patient group 1: birth to <1 year, and patient group 2: 1 to <12 years) were simulated. The optimal doses were simulated to match adult exposures in terms of total drug exposure (AUC_{inf}), C₂₄ and C₇₂, while not exceeding adult Cₘₐₓ. In the Japanese phase 2, global phase 3 studies and Japanese paediatric studies, baloxavir marboxil has shown a consistent and substantial drop in viral titres within 24 hours post dose. This supports the selection of C₂₄ as the primary PK metric for acute viral killing and use of this metric to inform exposure-matching to adults. However, because an adequate level of drug exposure beyond 24 hours may play a role to sustain inhibition of viral replication, model simulations also ensured the selected doses would adequately match adult exposure in terms of overall drug exposure (e.g., AUC_{inf} and C₇₂). A link between viral rebound and less sustained drug exposure over time (shorter T½ relative to adults) cannot be ruled out at this point.

Simulations of the anticipated drug exposure in non-Japanese paediatric subjects were obtained from the Japanese population PK model (Section 1.2, Table 5) with the following two optimizations:
(1) The disposition parameters CL/F and Vc/F obtained in Japanese paediatric patients were scaled by respectively 1.72 and 1.36, to account for the anticipated ethnic effect in these parameters as estimated from the global adult population PK model (Section 1.2, Table 4). A more detailed explanation of the factors 1.72 and 1.36 for accounting for the ethnic effect in pharmacokinetics of baloxavir marboxil can be found in Koshimichi, Hiroki, et al. “Population Pharmacokinetic and Exposure-Response Analyses of Baloxavir Marboxil in Adults and Adolescents Including Patients With Influenza.” Journal of pharmaceutical sciences (2018).
(2) A literature-based maturation factor (MF) was used to reduce CL/F parameter in an attempt to mimic ontogeny and select conservative doses in neonates and infants. MF was expressed as MF=PMAγ/(PMAγ+TM50γ), where PMA is postmenstrual age (weeks), TM50 is maturation half-life (54.2 weeks) until 50% maturation, and γ is Hill coefficient (3.92). The maturation factor (MF) is described, e.g. in Anderson, Paediatr Anaesth. 2011; 21: 222-37.

Model-based simulations (accounting for ethnic effect as well as bodyweight) indicated a regimen of 2 mg/kg up to 20 kg and 40 mg above 20 kg can be expected to mimic adult drug exposure adequately in terms of AUC_{inf}, C₂₄ and C₇₂ and was selected in paediatrics older than 3 months for studies CP40559 (Study 1) and CP40563 (Study 2). Furthermore, simulations confirm that this regimen can contain Cₘₐₓ below the current upper limit of exposure achieved and confirmed to be safe in humans so far. For younger infants (<3 months), where incomplete enzyme maturation cannot fully be ruled out to slightly reduce overall drug clearance, simulations are supportive that baloxavir marboxil dosing at 1 mg/kg is sufficient for adequate matching of drug exposure to adults.

Further details on the simulations which led to optimal doses for non-Asian (e.g. white such as Caucasian) paediatric patients are given in Examples 4 and 5, below.

### Example 4: Simulations for Optimal Doses for Non-Asian Pediatric Patients (1-12 Years Old)

The three dosing regimens explored were based on patient weight:
(1) 1 mg/kg<40 kg, 40 mg flat ≥40 kg (previously proposed regimen),
(2) 1.5 mg/kg<25 kg, 40 mg flat ≥25 kg, and
(3) 2.0 mg/kg<20 kg, 40 mg flat ≥20 kg.

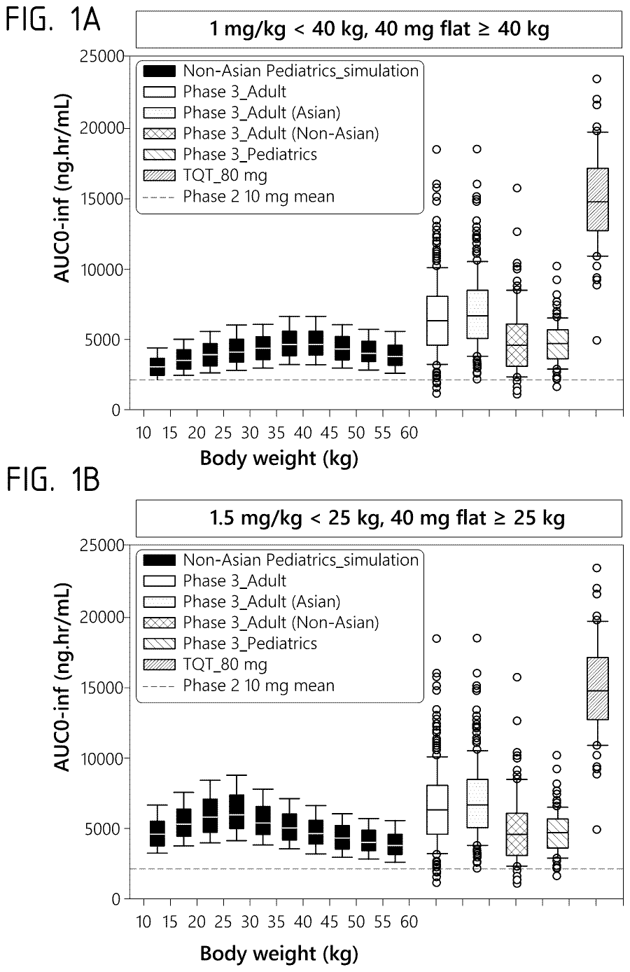

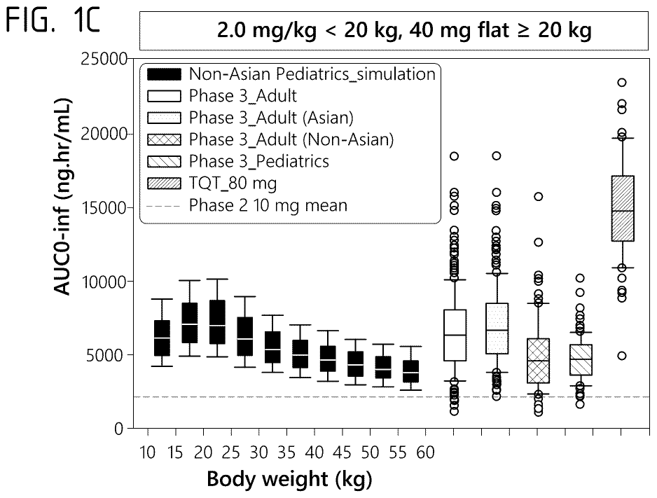

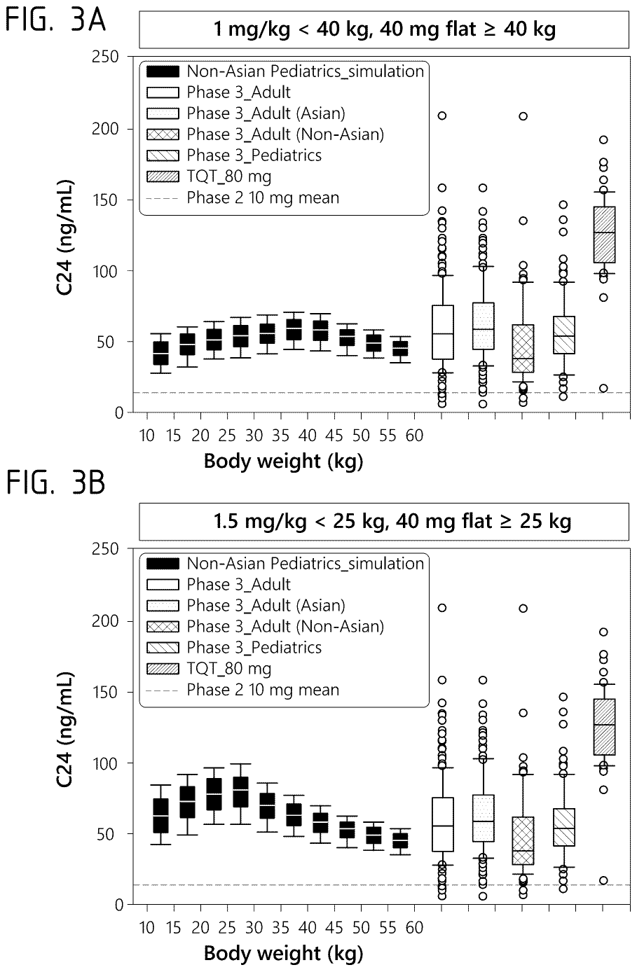

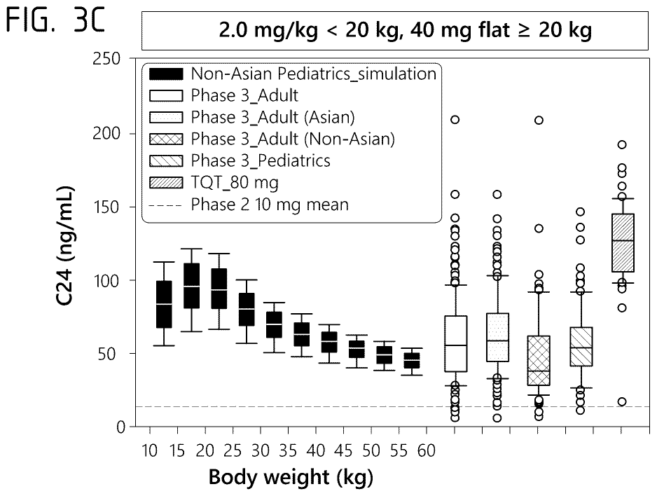

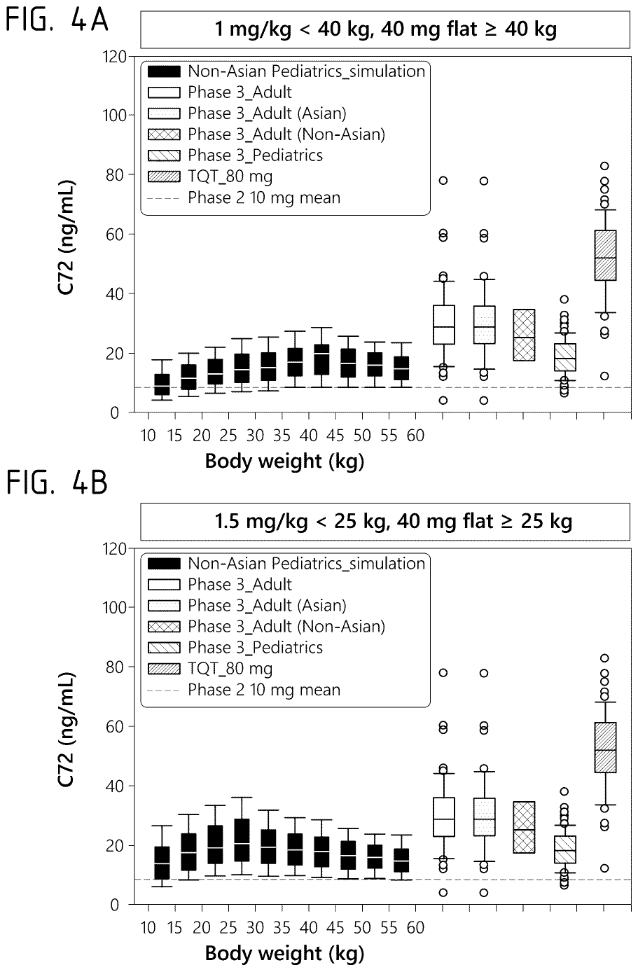

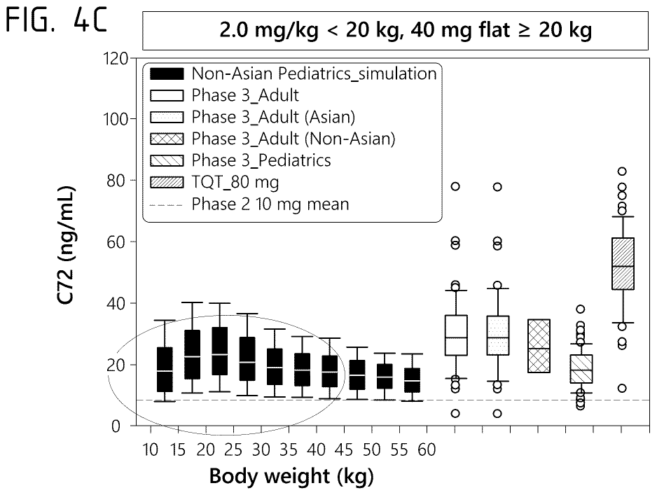

Of note, each regimen is tailored to the weight at which body weight (BW)-based dosing will stop, thereby managing risk to exceed 40 mg (adult reference dose). The projected pediatric drug exposure for various BW groups in terms of total drug exposure (AUC_{0-inf}), peak drug exposure (Cₘₐₓ), and drug concentration at 24 hours and 72 hours after dosing is depicted in FIGS. 1A-1C, FIGS. 2A-2C, FIGS. 3A-3C, and FIGS. 4A-4C, respectively. Adult reference exposure distributions for efficacy are shown for adults globally, and separated out for Caucasians and Asians. The thorough QT (TQT) study in Asians provides the current safe upper limit of exposure achieved in humans so far. In this regard, Q and T are two peaks in an electrocardiogram and if the distance between the two peaks changes during a clinical study it can indicate a drug's cardiac liability. In other words “TQT” measures side effects of the drug investigated on the heart (see, e.g., Grenier, Drug, healthcare and patient safety 10 (2018): 27).

As shown in previous studies, oral drug clearance of baloxavir is characterized to scale allometrically with an exponent of 0.632 on BW in Asian Pediatrics. Based on this relationship, BW-adjusted oral drug clearance (L/hr/kg) was estimated to be about 2-fold lower in a 10-kg child compared to an adult of 70 kg. In agreement with these calculations, the herewith provided simulation confirms that regimen three (i.e. regimen (3) shown above) matches adult exposure optimally in terms of both total drug exposure (FIGS. 1A-1C) as well as drug levels up to 72 hours after dosing (Error!Reference source not found.), particularly in pediatrics with a BW less than 25 kg. In light of the higher drug clearance and, hence, a faster disposition in children, a higher dose per BW (compared to adults) can also sustain drug exposure until at least 72 hours after dosing at similar levels as seen in adults. It can, however, be appreciated from Error!Reference source not found. that the improved exposure matching of regimen three (relative to regimen one) on AUC_{0-inf} and C72 comes at the expense of an increase in Cₘₐₓ (Error!Reference source not found.) and C₂₄ (Error!Reference source not found.) of about 2-fold (relative to regimen one). Nonetheless, average peak drug levels will remain below the levels measured in the adult thorough QT (TQT) study.

The optimal regimen should present the highest benefit-risk profile based on available data, and thus balances risks of compromised efficacy and safety. A single dose of baloxavir marboxil has been well tolerated in both adults and Asian pediatrics, and a substantial and consistent reduction in viral titers has been seen over a wide dose range, indicating a wide therapeutic window. In line with this wide window, no clear relationship has been found between drug exposure and occurrence of adverse events. Moreover, as baloxavir was well tolerated in the TQT study (with highest peak and total drug exposures so far achieved in human), it appears reasonable to consider the exposure data of this study as the best estimate of a safe upper limit of exposure in humans.

In the recently completed study using 1 mg/kg of baloxavir marboxil was used in Asian pediatrics weighing less than 20 kg. Our simulations support that more adequate exposure matching to adults can be achieved in terms of both total (AUC_{0-inf}) and sustained (C₇₂) drug exposure using either regimen two or three (i.e. regimen (2) or (3) specified above). Regimen three however mimics adult exposure better than regimen two, while both regimens can contain exposure with sufficient confidence within a reliable benchmark shown to be safe in adults.

Of note, in addition to our simulations, the available sparse PK data in the recently completed Asian pediatric study 1602T0833 (enrolling pediatrics weighing less than 20 kg) confirms drug concentrations briefly after dosing to fluctuate at about the mean of 100 ng/mL (1 mg/kg). Since PK is known to be linear with dose, a dose of 2 mg/kg can be expected to increase exposure by a factor of 2. It appears reasonable therefore to propose regimen three (i.e. regimen (3) specified above): 2 mg/kg for patients weighing up to 20 kg (and 40 mg flat for patients weighing more than 20 kg).

### Example 5: Simulations for Optimal Doses for Non-Asian Pediatric Patients (0-1 Year Old)

The three dosing regimens explored were:
(1) 1 mg/kg (previously proposed regimen),
(2) 1.5 mg/kg, and
(3) 2.0 mg/kg.

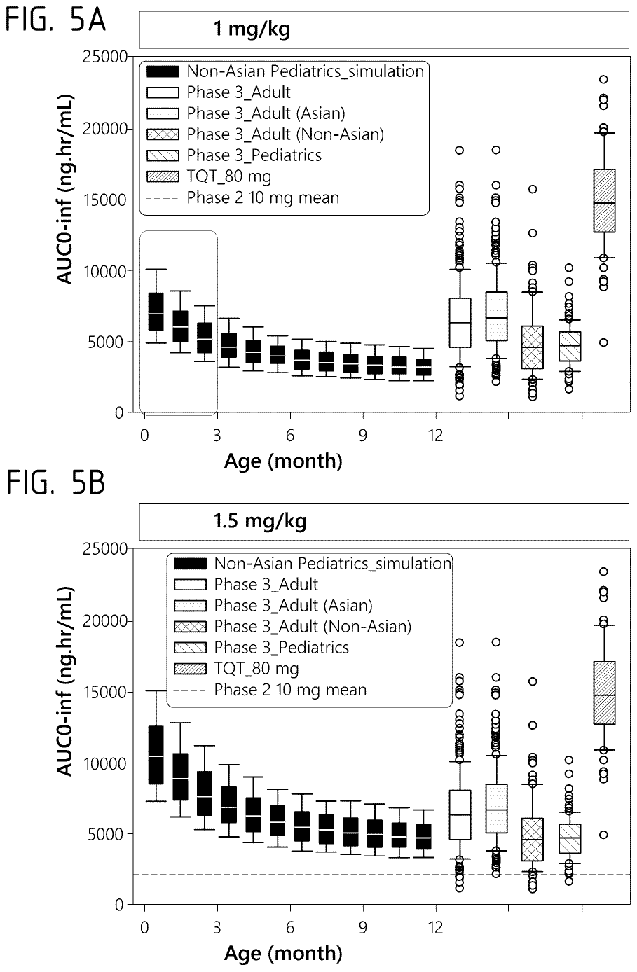

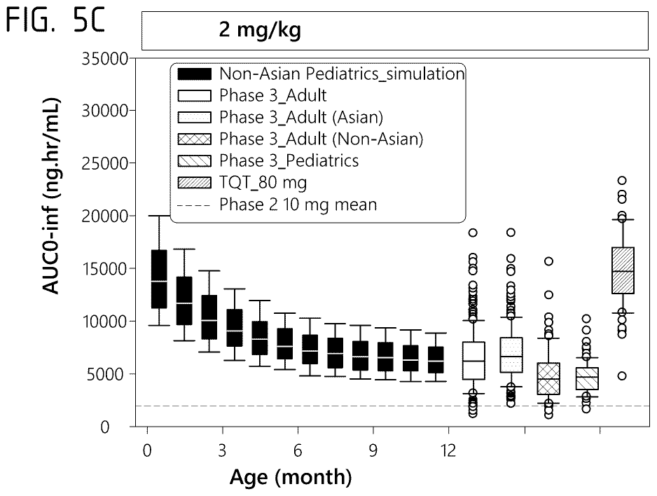

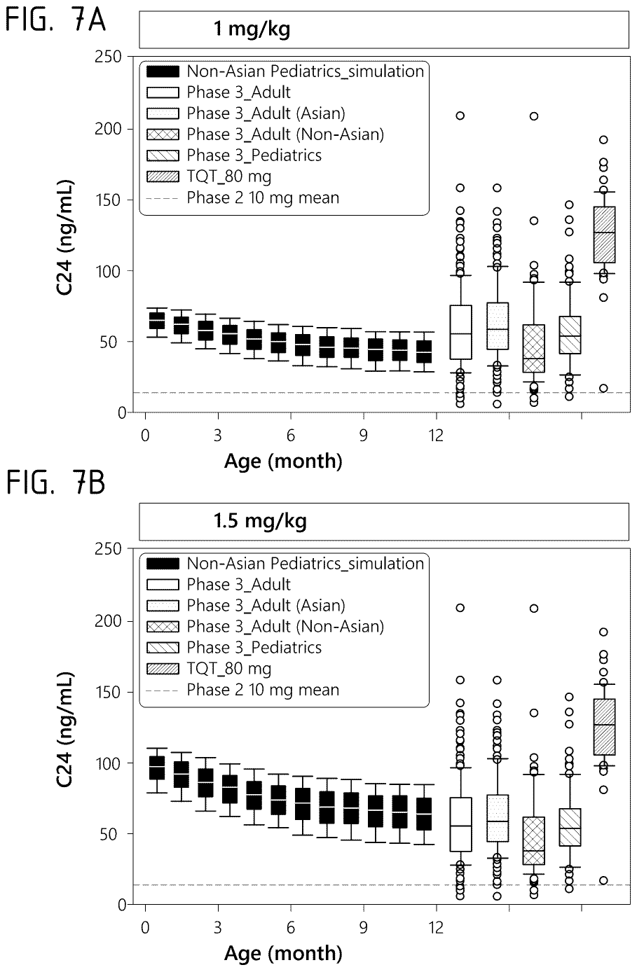

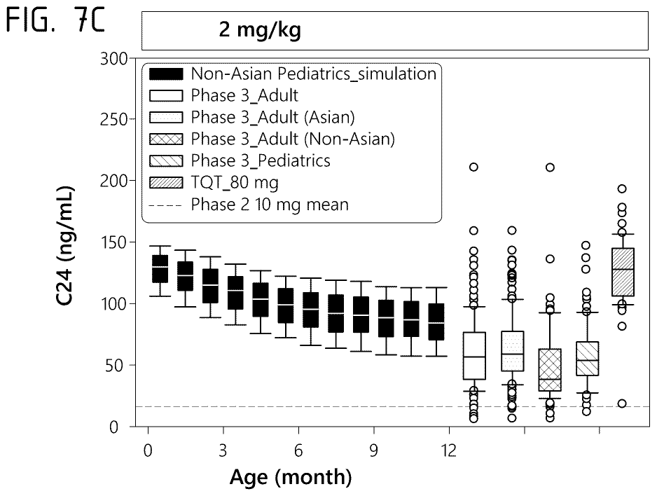

Simulation of pediatric drug exposure distribution in terms of AUC_{0-inf}, Cₘₐₓ, C₂₄, and C₇₂ are depicted in FIGS. 5A-5C, FIGS. 6A-6C, FIGS. 7A-7C, and FIGS. 8A-8C, respectively.

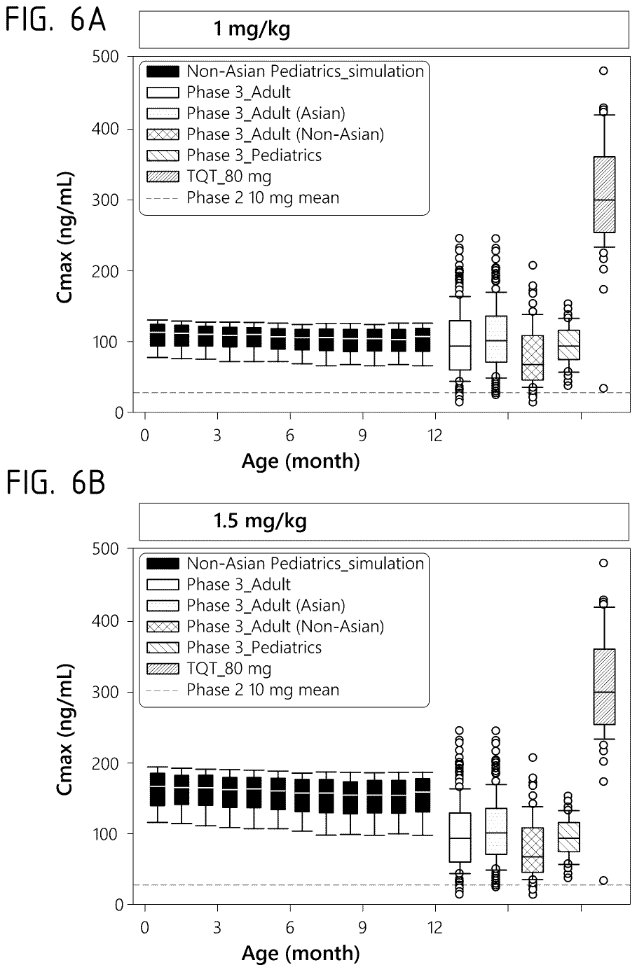

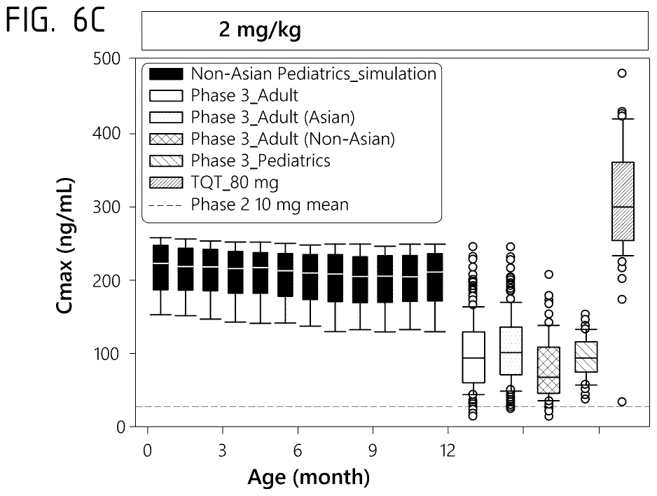

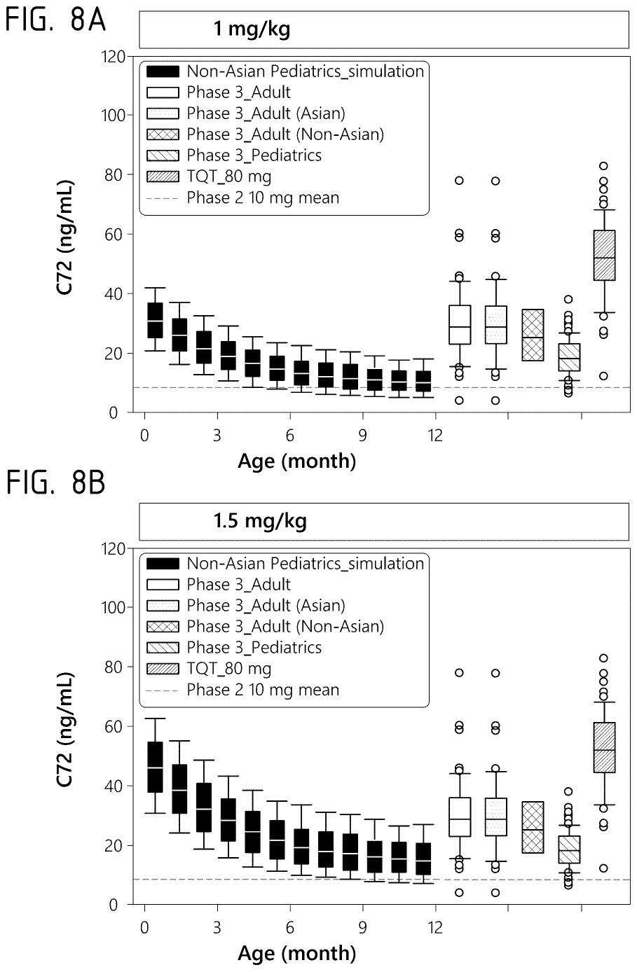

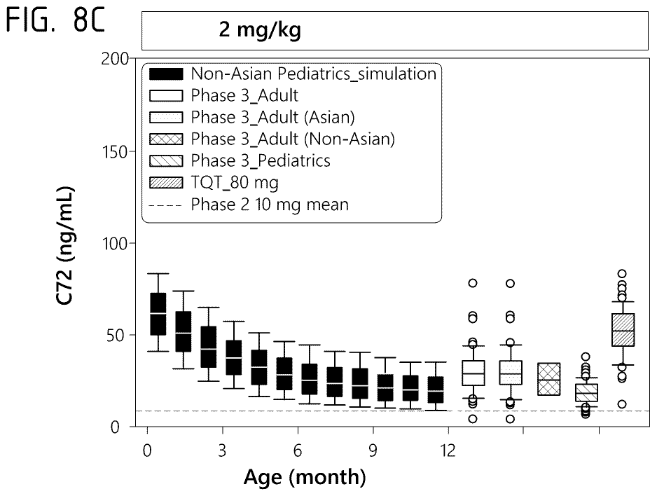

In agreement with the simulations for 1-12 year old children, regimen three (i.e. regimen (3) specified above) matches adult exposure most optimally in terms of total drug exposure (AUC_{0-inf}) and C₇₂, at least for infants aged 3 months and older. For the younger infants (<3 months), where incomplete enzyme maturation might slightly reduce overall drug clearance, simulations are supportive that baloxavir marboxil dosing at 1 mg/kg is sufficient for adequate matching of AUC_{0-inf} to adults. In infants older than 3 months, the overall increase in AUC_{0-inf} using regimen three is also expected to improve matching of drug exposure to adults in terms of C₇₂ (FIGS. 8A-8C), but with an approximate 2-fold increase in terms of Cₘₐₓ compared to regimen one (FIGS. 6A-6C). Of note, since baloxavir has low oral drug clearance, in addition to a demonstrated age-independent absolute bio-availability (similar Cₘₐₓ in adults and children seen in Asian patients at 1 mg/kg), Cₘₐₓ predictions can be made with fairly high confidence across age-groups (note also that the volume of distribution is demonstrated to be proportional to BW).

Taken together, these simulations support the ability to improve benefit-risk assessment for infants of 3 months and older with a regimen of 2 mg/kg, while the reduced dose of 1 mg/kg is considered sufficient for younger infants (4 weeks-3 months) as well as for newborns (0-4 weeks).

### Example 6: Preparation of Granulae Comprising the Compound of the Invention

### A. Preparation of Granulae Compositions

A compound II can be produced, e.g., by a method disclosed in International Publication No. WO 2016/175224.

### Manufacturing Method for Compound I

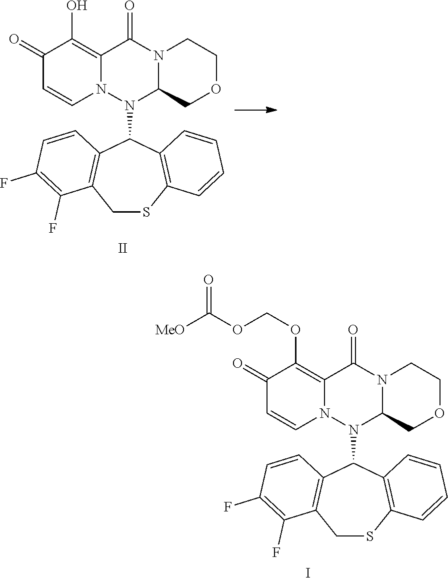

Potassium carbonate (1483.4 mg, 10.7 mmol), potassium iodide (549.5 mg, 3.3 mmol), tetrahydrofuran (33.1 g), N,N-dimethylacetamide (3.8 g) and water (80.3 mg) were added to the compound II (4.0 g, 8.3 mmol), followed by stirring. The resultant mixture was heated to 60° C., to which chloromethyl methyl carbonate (1758.9 mg, 14.2 mmol) was added. The resultant was stirred at 60° C. for 9 hours, and then cooled to 20° C. Acetic acid (822.0 mg), 2-propanol (3.1 g) and water (20.0 g) were added thereto, and the resultant was extracted twice with tetrahydrofuran (1.8 g, 8.9 g). The solvent was distilled off through vacuum concentration to a liquid weight of about 32 g. The resultant was heated to 45° C., 2-propanol (1.6 g) was added thereto, and the resultant was cooled to 20° C. A sodium acetate aqueous solution prepared from sodium acetate (339.0 mg) and water (46.0 g) was added thereto, followed by cooling to 5° C. After the resultant was stirred at 5° C. for 3 hours, a pale yellow precipitate was filtered off. The thus obtained solid was washed with a mixture of 2-propanol (4.7 g) and water (6.0 g), and the solid was then washed again with 2-propanol (6.3 g). To the thus obtained pale yellow solid, dimethyl sulfoxide (30.9 g) was added, followed by stirring. The resultant was heated to 60° C., to which a mixture of dimethyl sulfoxide (2.2 g) and water (4.8 g) was added. A mixture of dimethyl sulfoxide (19.9 g) and water (28.4 g) was further added thereto, followed by cooling to 20° C. After the resultant was stirred at 20° C. for 3 hours, a generated white precipitate was filtered off. The thus obtained solid was washed with a mixture of dimethyl sulfoxide (8.0 g) and water (4.8 g), and the solid was washed again with water (12.0 g). The thus obtained solid was dried to give a compound I (4.21 g) as white crystal.

¹H-NMR (DMSO-D6) δ: 2.91-2.98 (1H, m), 3.24-3.31 (1H, m), 3.44 (1H, t, J=10.4 Hz), 3.69 (1H, dd, J=11.5, 2.8 Hz), 3.73 (3H, s), 4.00 (1H, dd, J=10.8, 2.9 Hz), 4.06 (1H, d, J=14.3 Hz), 4.40 (1H, d, J=11.8 Hz), 4.45 (1H, dd, J=9.9, 2.9 Hz), 5.42 (1H, dd, J=14.4, 1.8 Hz), 5.67 (1H, d, J=6.5 Hz), 5.72-5.75 (3H, m), 6.83-6.87 (1H, m), 7.01 (1H, d, J=6.9 Hz), 7.09 (1H, dd, J=8.0, 1.1 Hz), 7.14-7.18 (1H, m), 7.23 (1H, d, J=7.8 Hz), 7.37-7.44 (2H, m) Powder X-ray Diffraction: 2θ(°): Characteristic peaks are present at 8.6°±0.2°, 14.1°±0.2°, 17.4°±0.2°, 20.0°±0.2°, 24.0°±0.2°, 26.3°±0.2°, 29.6°±0.2° and 35.4°±0.2°.

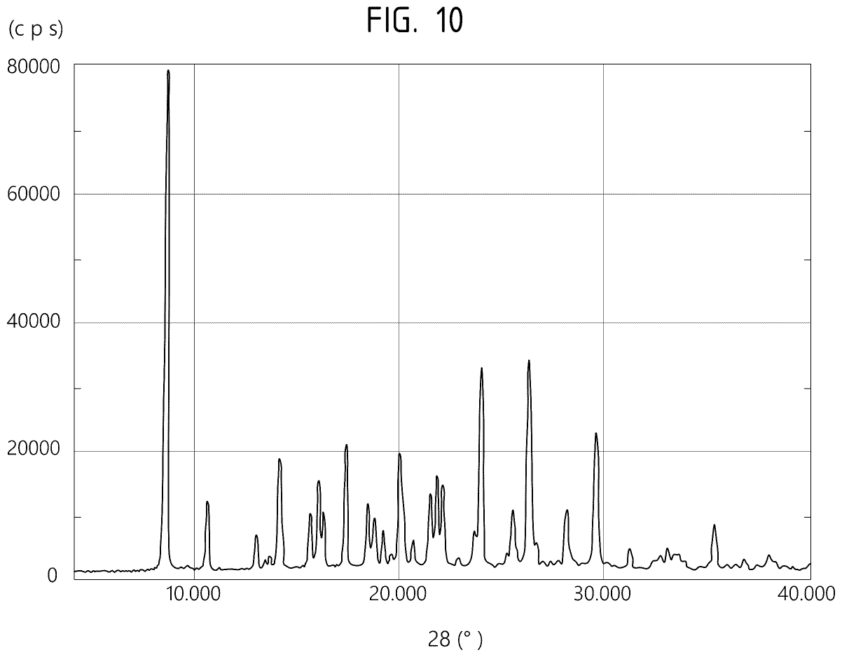

The powder X-ray diffraction pattern of the crystal of compound I is shown in FIG. 10.

### (1) Study on Stabilizer

In order to study a stabilizer, a stabilizer shown in each of Tables 7 to 9 and a compound represented by formula (I) were wet-granulated, and the amount of increase in the compound represented by formula (II), which is a related substance, were evaluated after a temporal stability test of the produced granule. A preparation having a formulation shown in Table 6 was produced by the stirring granulation method.

TABLE 6

| | Content (mg) |
|---|---|
| Compound represented by Formula (I) | 2.0 |
| Purified White Sugar | 488.0 |
| Hydrogenated Maltose Starch Syrup (Maltitol) | 500.0 |
| Stabilizer | 30.0 |
| Hydroxypropyl Cellulose | 10.0 |
| Total | 1030.0 |

### (Method for Manufacturing Preparation)

A compound represented by formula (I), purified white sugar, powdered hydrogenated maltose starch syrup (maltitol), a stabilizer and hydroxypropyl cellulose shown in Table 6 were mixed using a high-speed mixer (FS-GS SJT 10 high-speed mixer, Fukae Powtec Co., Ltd.), and water was added to the mixture, followed by stirring granulation. Then, the granulation product was subjected to size selection in a power mill (model P-3S, Showa Kagakukikai Co., Ltd.), and the resultant was dried at 65 to 70° C. in a fluidized bed granulator (WSG2&5 fluid bed dryer granulator, Okawara Mfg. Co., Ltd.). After drying, a granule was obtained by size selection in a power mill (model P-3S, Showa Kagakukikai Co., Ltd.). Granulation conditions in the high-speed mixer were as follows:

### (Granulation Conditions)

Granulator: FS-GS SJT 10 high-speed mixer
Rotational Speed of Agitator: 250 rpm
Rotational Speed of Chopper: 2500 rpm
Acceleration in Solution Injection: 21±2 g/min
Moisture: 4 to 6.5% by weight
Mashing time: 1 min±5 sec

### (Temporal Stability Test of Preparation)

The produced preparation was stored at 60° C. for 2 weeks, and the amount of increase in the compound represented by formula (II), which is a related substance, was measured.

### (Stabilizer)

As shown in Tables 7 to 9, sodium chloride (Kanto Chemical Co., Inc.), potassium chloride (Wako Pure Chemical Industries, Ltd.), ascorbic acid (Nacalai Tesque, Inc.), fumaric acid (Merck KGaA), medium-chain fatty acid triglyceride Miglyol (Mitsuba Trading Co., Ltd.), triethyl citrate (Merck KGaA), sodium nitrite (Nacalai Tesque, Inc.), glycerin (Kanto Chemical Co., Inc.), and vitamin E (Merck KGaA) were used as the stabilizer.

TABLE 7

| | Example 7-1 | Example 7-2 | Example 7-3 | Example 7-4 |
|---|---|---|---|---|
| Stabilizer | Sodium | Potassium | Ascorbic | Fumaric |
| | Chloride | Chloride | Acid | Acid |

TABLE 8

| | | | Comparative | Comparative |
|---|---|---|---|---|
| | Example 7-5 | Example 7-6 | Example 7-1 | Example 7-2 |
| Stabilizer | Medium-Chain Fatty Acid | Triethyl Citrate | Sodium Nitrite | Glycerin |
| | Triglyceride Miglyol | | | |

TABLE 9

| | Comparative | Comparative |
|---|---|---|
| | Example 7-3 | Example 7-4 |
| Stabilizer | Vitamin E | None |

(Method for Measuring Compound represented by Formula (II))

The amount of the compound represented by formula (II) was measured by liquid chromatography by employing the following method and conditions:
Detector: ultraviolet absorptiometer (measurement wavelength: 260 nm)
Column: XBridge C18, 3.5 μm, 3.0×150 mm
Column temperature: constant temperature around 35° C.
Mobile Phase A: 0.1% trifluoroacetic acid/0.2 mM EDTA solution, Mobile Phase B: acetonitrile
Delivery of mobile phase: controlled for a concentration gradient with a mixing ratio between the mobile phase A and the mobile phase B changed as shown in Table 10.

TABLE 10

| | Mobile Phase | Mobile Phase |
|---|---|---|
| Time after Injection (min) | A (vol %) | B (vol %) |
| 0-5 | 70 | 30 |
| 5-40 | 70 → 20 | 30 → 80 |
| 40-40.1 | 20 → 70 | 80 → 30 |

Flow rate: about 0.6 mL/min
Injection amount: 5 μL
Sample cooler temperature: about 5° C.
Washing solution for autoinjector: acetonitrile/methanol mixture (1:3)
Range of area measurement: 50 minutes after injection of sample solution
Equation for calculating amount of compound represented by formula (II):

Amount of compound represented by formula (II)(%)=(ATII/ΣA_{T})×100
ATII: peak area of compound represented by formula (II) in sample solution
ΣA_{T}: Sum of peak areas of sample solution (excluding blank and system peaks)

### (Results)

The amount of increase (%) in the compound represented by formula (II) in the temporal stability test of the preparations of Examples 7-1 to 7-6 and Comparative Examples 7-1 to 7-4 is shown in Tables 11 to 13. As a result, the amount of increase (%) in the compound represented by formula (II) in the granules of Examples 7-1 to 7-6 was lower than that in the granule containing no stabilizer of Comparative Example 7-4. Particularly, the amount of increase in the compound represented by formula (II) in the granules containing sodium chloride of Example 7-1, ascorbic acid of Example 7-3, fumaric acid of Example 7-4 and medium-chain fatty acid triglyceride Miglyol of Example 7-5 was much smaller than that in the granule containing no stabilizer of Comparative Example 7-4.

TABLE 11

| | Example 7-1 | Example 7-2 | Example 7-3 | Example 7-4 |
|---|---|---|---|---|
| Stabilizer | Sodium | Potassium | Ascorbic | Fumaric |
| | Chloride | Chloride | Acid | Acid |
| Amount of Increase (%) | 0.70 | 1.31 | 0.28 | 0.30 |
| in Compound represented | | | | |
| by Formula (II) | | | | |

TABLE 12

| | | | Comparative | Comparative |
|---|---|---|---|---|
| | Example 7-5 | Example 7-6 | Example 7-1 | Example 7-2 |
| Stabilizer | Medium-Chain Fatty Acid | Triethyl | Sodium Nitrite | Glycerin |
| | Triglyceride Miglyol | Citrate | | |
| Amount of Increase (%) | 0.34 | 1.24 | 6.63 | 9.95 |
| in Compound represented | | | | |
| by Formula (II) | | | | |

TABLE 13

| | Comparative | Comparative |
|---|---|---|
| | Example 7-3 | Example 7-4 |
| Stabilizer | Vitamin E | None |
| Amount of Increase (%) | 3.56 | 1.35 |
| in Compound represented | | |
| by Formula (II) | | |

### (2) Study on Excipient

In order to study an excipient, an excipient shown in each of Tables 14 to 16 and a compound represented by formula (I) were wet-granulated, and the amount of increase in the compound represented by formula (II), which is a related substance, was evaluated after a temporal stability test of the produced granule.

### (Method for Producing Preparation)

An excipient shown in each of Tables 14 to 16 and a compound represented by formula (I) were mixed in a bag at a ratio of 1:1, and then, the mixture was sieved through a 30-mesh sieve (wire diameter: 0.22 mm). The sieved mixed powder was mixed in a mortar, and then, purified water was gradually added such that moisture in granulation was about 5% by weight based on the charged amount of the materials, and the resultant was kneaded using a pestle. The kneaded product was subjected to wet size selection while pressed by hand through 16-mesh wires (wire diameter: 0.55 mm). The granulation product after the size selection was dried in a vented dryer, and a granule was prepared while pressed by hand through 20-mesh wires (wire diameter: 0.40 mm).

### (Temporal Stability Test of Preparation)

The produced preparation was stored at 60° C. for 2 weeks, and the amount of increase in the compound represented by formula (II), which is a related substance, was measured.

### (Excipient)

As shown in Tables 14 to 16, purified white sugar (Merck KGaA), hydrogenated maltose starch syrup (maltitol, ROQUETTE), D-mannitol (ROQUETTE), lactose hydrate (DMV-Fonterra Excipients GmbH & Co. KG), sorbitol (Merck KGaA), erythritol (ROQUETTE), xylitol (ROQUETTE), and isomalt (Beneo-Palatinit GmbH) were used as the excipient

TABLE 14

| | Example 7-7 | Example 7-8 | Example 7-9 |
|---|---|---|---|
| Excipient | Purified White Sugar | Hydrogenated Maltose | D-Mannitol |
| | | Starch Syrup (Maltitol) | |

TABLE 15

| | Reference | Reference | Reference |
|---|---|---|---|
| | Example 7-1 | Example 7-2 | Example 7-3 |
| Excipient | Lactose Hydrate | Sorbitol | Erythritol |

TABLE 16

| | Reference | Reference |
|---|---|---|
| | Example 7-4 | Example 7-5 |
| Excipient | Xylitol | Isomalt |

### (Results)

The amount of increase (%) in the compound represented by formula (II) in the temporal stability test of the preparations of Examples 7-7 to 7-9 and Reference Examples 7-1 to 7-5, and the melting point of each excipient are shown in Tables 17 to 19. As a result, the amount of increase (%) in the compound represented by formula (II) in the granules of Examples 7-7 to 7-9 was slightly lower than that in the granules of Reference Examples 7-1, 7-2 and 7-5. The amount of increase (%) in the compound represented by formula (II) in the granules of Reference Examples 7-3 and 7-4 was almost the same as that in the granules of Examples 7-7 to 7-9, whereas the melting point was lower as compared with Examples 7-7 to 7-9 and thus, there was a possibility of sticking. Accordingly, it was regarded that purified white sugar, hydrogenated maltose starch syrup (maltitol) and D-mannitol are preferred as the excipient.

TABLE 17

| | Example 7-7 | Example 7-8 | Example 7-9 |
|---|---|---|---|
| Excipient | Purified White Sugar | Hydrogenated Maltose | D-Mannitol |
| | | Starch Syrup (Maltitol) | |
| Melting point (° C.) | 160-186 | 145 | 166-168 |
| Amount of Increase (%) | 0.08 | 0.06 | 0.11 |
| in Compound represented | | | |
| by Formula (II) | | | |

TABLE 18

| | Reference | Reference | Reference |
|---|---|---|---|
| | Example 7-1 | Example 7-2 | Example 7-3 |
| Excipient | Lactose Hydrate | Sorbitol | Erythritol |
| Melting point (° C.) | 201-202 | 95 | 121 |
| Amount of Increase (%) | 0.17 | 0.15 | 0.08 |
| in Compound represented | | | |
| by Formula (II) | | | |

TABLE 19

| | Reference | Reference |
|---|---|---|
| | Example 7-4 | Example 7-5 |
| Excipient | Xylitol | Isomalt |
| Melting point (° C.) | 92-96 | 141-161 |
| Amount of Increase (%) | 0.04 | 0.38 |
| in Compound represented | | |
| by Formula (II) | | |

### (3) Study on Combination of Excipients

Although purified white sugar, hydrogenated maltose starch syrup (maltitol) and D-mannitol were selected as a preferable excipient, in order to study a combination of these excipients, a combination of excipients shown in each of Tables 20 and 21 and a compound represented by formula (I) were wet-granulated, and the produced granule was evaluated for (a) the amount of increase in the compound represented by formula (II), which is a related substance, (b) suspensibility in water, (c) container adherence, (d) a fine granule yield, and (e) a bulk density. A preparation having a formulation shown in each of Tables 20 and 21 was produced by the stirring granulation method.

TABLE 20

| | Example 7-10 | Example 7-11 | Example 7-12 |
|---|---|---|---|
| | (weight mg) | (weight mg) | (weight mg) |
| Compound | 10.0 | 20.0 | 10.0 |
| represented by | | | |
| Formula (I) | | | |
| Maltitol | 300.0 | 350.0 | 490.0 |
| D-Mannitol | 614.0 | 554.0 | 490.0 |
| Purified White Sugar | — | — | — |
| Sodium Chloride | 30.0 | 30.0 | — |
| Polyvinyl Pyrrolidone | 10.0 | 10.0 | 10.0 |
| k25 | | | |
| Total | 964.0 | 964.0 | 1000.0 |
| Weight Ratio of Sugar | Maltitol:D- | Maltitol:D- | Maltitol:D- |
| or Sugar Alcohol | Mannitol = | Mannitol = | Mannitol = |
| | 32.8:67.2 | 38.7:61.3 | 50.0:50.0 |

TABLE 21

| | Comparative | Comparative |
|---|---|---|
| | Example 7-5 | Example 7-6 |
| | (weight mg) | (weight mg) |
| Compound | 10.0 | 10.0 |
| represented by | | |
| Formula (I) | | |
| Maltitol | 500.0 | — |
| D-Mannitol | — | 500.0 |
| Purified White Sugar | 480.0 | 480.0 |
| Sodium Chloride | — | — |
| Polyvinyl Pyrrolidone | 10.0 | 10.0 |
| k25 | | |
| Total | 1000.0 | 1000.0 |
| Weight Ratio of Sugar | Maltitol:Purified | D-Mannitol:Purified |
| or Sugar Alcohol | White Sugar = | White Sugar = |
| | 51.0:49.0 | 51.0:49.0 |

### (Method for Producing Preparation)

A compound represented by formula (1), an excipient and polyvinyl pyrrolidone shown in each of Tables 20 and 21 were mixed using a high-speed mixer (LFS-GS-2J high-speed mixer, Fukae Powtec Co., Ltd.), and water was added to the mixture, followed by stirring granulation. Then, the granulation product was subjected to size selection in a power mill (model P-3S, Showa Kagakukikai Co., Ltd.), and the resultant was dried at 65 to 70° C. in a fluidized bed granulator (MP-01 Fluid bed dryer granulator, Powrex Corp.). After drying, a granule was obtained by size selection in a power mill (model P-3S, Showa Kagakukikai Co., Ltd.). Granulation conditions in the high-speed mixer were as follows:

### (Granulation Conditions)

Granulator: LFS-GS-2J high-speed mixer
Rotational Speed of Agitator: 333 rpm
Rotational Speed of Chopper: 2500 rpm
Acceleration in Solution Injection: 20±3.5 g/min
Moisture: 3 to 7.5% by weight
Mashing time: 1 to 2 min±5 sec

### (Suspensibility Test of Preparation in Water)

The number of times of mix by inversion required for preparing a visually uniform suspension when 9.5 mL of water was added to about 1 g of the present preparation was recorded.

### (Container Adherence of Preparation)

In the production of the present preparation, the amount of a granulation product adhering to the interior wall of a stirring granulator after granulation was visually confirmed. The presence or absence of adhesion after scraping off was evaluated as an index for container adherence.

### (Fine Granule Yield Measurement of Preparation)

100 g of the present preparation was sieved through Nos. 30 and 140 sieves, and the ratio of the amount of a granule passing through the No. 30 sieve and remaining on the No. 40 sieve to the total amount of the sieved granule was calculated.

### (Bulk Density Measurement of Preparation)

The present preparation was injected to a container (capacity: 100 mL) until overflowing, and the preparation was carefully leveled off to remove an excess from the upper surface of the container. The value of a preparation weight in the container was obtained from a container weight tared in advance, and a bulk density was determined according to the following equation:

$$Bulk density = Preparation weight in container / 100$$

### (Excipient)

As shown in Tables 20 and 21, purified white sugar (Merck KGaA), hydrogenated maltose starch syrup (maltitol, ROQUETTE), and D-mannitol (ROQUETTE) were used in combination as the excipient.

### (Results)

The suspensibility in water, container adherence, fine granule yield and bulk density of the preparations of Examples 7-10 to 7-12 and Comparative Examples 7-5 and 7-6 are shown in Tables 22 and 23. As a result, the preparations of Examples 7-10 to 7-12 containing a mixture of hydrogenated maltose starch syrup (maltitol) and D-mannitol as an excipient had excellent suspensibility in water, small adherence to a container, and a bulk density of 0.5 g/mL or larger. Particularly, in Examples 7-10 and 7-11, the fine granule yield was also as high as 90% or more. On the other hand, the preparations of Comparative Examples 7-5 and 7-6 containing a mixture of purified white sugar and hydrogenated maltose starch syrup (maltitol) or purified white sugar and D-mannitol as an excipient were inferior in suspensibility in water to Examples and also had large container adherence. Particularly, in Comparative Example 7-6, the fine granule yield was also low.

TABLE 22

| | Example 7-10 | Example 7-11 | Example 7-12 |
|---|---|---|---|
| Suspensibility | Uniformly | Uniformly | Uniformly |
| in Water | suspended by | suspended by | suspended by |
| | 15 times | 10 times | 10 times |
| Container Adherence | Small | Small | Small |
| Fine Granule Yield | 92 | 90 | 72 |
| (%) | | | |
| Bulk Density (g/mL) | 0.67 | 0.67 | 0.59 |

TABLE 23

| | Comparative | Comparative |
|---|---|---|
| | Example 7-5 | Example 7-6 |
| Suspensibility | Uniformly | Uniformly |
| in Water | suspended by | suspended by |
| | 25 times | 30 times |
| Container Adherence | Large | Large |
| Fine Granule Yield | 89 | 66 |
| (%) | | |
| Bulk Density (g/mL) | 0.76 | 0.65 |

### (4) Study on Binder

In order to study a binder, a binder shown in Table 24 and a compound represented by formula (I) were wet-granulated, and the produced preparation was evaluated for (a) the amount of increase in the compound represented by formula (II), which is a related substance, after a temporal stability test and (b) a bulk density. A preparation having a formulation shown in Table 24 was produced by the stirring granulation method. Polyvinyl pyrrolidone K25 (BASF) and hydroxypropyl cellulose SL (Shin-Etsu Chemical Co., Ltd.) were used as the binder.

TABLE 24

| | | | Reference |
|---|---|---|---|
| | Example 7-13 | Example 7-14 | Example 7-6 |
| | (weight mg) | (weight mg) | (weight mg) |
| Compound represented | 10.0 | 10.0 | 10.0 |
| by Formula (I) | | | |
| Purified White Sugar | 480.0 | 460.0 | 480.0 |
| Hydrogenated Maltose | 500.0 | 500.0 | 500.0 |
| Starch syrup (Maltitol) | | | |
| Polyvinyl Pyrrolidone | 10.0 | 30.0 | — |
| K25 | | | |
| Hydroxypropyl Cellulose | — | — | 10.0 |
| SL | | | |
| Total | 1000.0 | 1000.0 | 1000.0 |

### (Method for Producing Preparation)

A compound represented by formula (I), purified white sugar, hydrogenated maltose starch syrup (maltitol), and hydroxypropyl cellulose SL (Nippon Soda Co., Ltd.) or polyvinyl pyrrolidone K25 as a binder shown in Table 24 were mixed using a high-speed mixer (LFS-GS-2J high-speed mixer, Fukae Powtec Co., Ltd.), and water was added to the mixture, followed by stirring granulation. Then, the granulation product was subjected to size selection in a power mill (model P-3S, Showa Kagakukikai Co., Ltd.), and the resultant was dried at 65 to 70° C. in a fluidized bed granulator (MP-01 Fluid bed dryer granulator, Powrex Corp.). After drying, a granule was obtained by size selection in a power mill (model P-3S, Showa Kagakukikai Co., Ltd.). Granulation conditions in the high-speed mixer were as follows:

### (Granulation Conditions)

Granulator: LFS-GS-2J high-speed mixer
Rotational Speed of Agitator: 333 rpm
Rotational Speed of Chopper: 2500 rpm
Acceleration in Solution Injection: 20±3.5 g/min
Moisture: 3 to 7.5% by weight
Mashing time: 1 to 2 min±5 sec

### (Temporal Stability Test of Preparation)

The produced preparation was stored at 60° C. for 2 weeks, and the amount of increase in the compound represented by formula (II), which is a related substance, was measured.

### (Bulk Density Measurement of Preparation)

The present preparation was injected to a container (capacity: 100 mL) until overflowing, and the preparation was carefully leveled off to remove an excess from the upper surface of the container. The value of a preparation weight in the container was obtained from a container weight tared in advance, and a bulk density was determined according to the following equation:

$$Bulk density = Preparation weight in container / 100$$

### (Results)

The amount of increase (%) in the compound represented by formula (II) in the temporal stability test of the preparations of Examples 7-13 and 7-14 and Reference Example 7-6, and the bulk density are shown in Table 25. As a result, the amount of increase (%) in the compound represented by formula (II) in the preparations of Examples 7-12 and 7-13 containing polyvinyl pyrrolidone was lower than that in the preparation of Reference Example 7-6 containing hydroxypropyl cellulose. The amount of increase (%) in the compound represented by formula (II) in the temporal stability test and the bulk density in the preparation of Example 7-12 in which the amount of polyvinyl pyrrolidone was 1% by weight were lower than those in the preparation of Example 7-13 in which the amount of polyvinyl pyrrolidone was 3% by weight.

TABLE 25

| | | | Reference |
|---|---|---|---|
| | Example 7-13 | Example 7-14 | Example 7-6 |
| Amount of Increase | 0.12 | 0.15 | 0.20 |
| (%) in Compound | | | |
| represented by Formula | | | |
| (II) | | | |
| Bulk Density (g/mL) | 0.72 | 0.77 | — |

### (5) Study on Fluidizing Agent

In order to study a fluidizing agent, (a) the amount of related substances after temporal storage of a preparation and (b) stickiness between preparations were evaluated. A preparation having a formulation shown in each of Tables 26 and 27 was produced by the stirring granulation method. 1% and 3% light anhydrous silicic acid (Cab-o-sil, Cabot Corp.), 1% and 3% hydrated silicon dioxide (RxCIPIENTS) and 1% and 3% sodium stearyl fumarate (PRUV, JRS Pharma) were used as the fluidizing agent.

TABLE 26

| | Example 7-15 | Example 7-16 | Example 7-17 |
|---|---|---|---|
| | (weight mg) | (weight mg) | (weight mg) |
| Compound represented | 10.0 | 10.0 | 10.0 |
| by Formula (I) | | | |
| Hydrogenated Maltose | 490.0 | 490.0 | 490.0 |
| Starch Syrup (Maltitol) | | | |
| D-Mannitol | 490.0 | 490.0 | 490.0 |
| Polyvinyl Pyrrolidone | 10.0 | 10.0 | 10.0 |
| k25 | | | |
| Sucralose | 5.0 | 5.0 | 5.0 |
| Light Anhydrous | 10.0 | 30.0 | — |
| Silicic Acid | | | |
| Hydrated Silicon | — | — | 10.0 |
| Dioxide | | | |
| Sodium Stearyl | — | — | — |
| Fumarate | | | |
| Strawberry Flavor | 1.0 | 1.0 | 1.0 |
| Total | 1016.0 | 1036.0 | 1016.0 |

TABLE 27

| | | Comparative | Comparative |
|---|---|---|---|
| | Example 7-18 | Example 7-7 | Example 7-8 |
| | (weight mg) | (weight mg) | (weight mg) |
| Compound represented | 10.0 | 10.0 | 10.0 |
| by Formula (I) | | | |
| Hydrogenated Maltose | 490.0 | 490.0 | 490.0 |
| Starch Syrup (Maltitol) | | | |
| D-Mannitol | 490.0 | 490.0 | 490.0 |
| Polyvinyl Pyrrolidone | 10.0 | 10.0 | 10.0 |
| k25 | | | |
| Sucralose | 5.0 | 5.0 | 5.0 |
| Light Anhydrous Silicic | — | — | 10.0 |
| Acid | | | |
| Hydrated Silicon Dioxide | 30.0 | — | — |
| Sodium Stearyl Fumarate | — | 10.0 | 30.0 |
| Strawberry Flavor | 1.0 | 1.0 | 1.0 |
| Total | 1036.0 | 1016.0 | 1036.0 |

### (Method for Producing Preparation)

A compound represented by formula (I), hydrogenated maltose starch syrup (maltitol), D-mannitol, polyvinyl pyrrolidone K25, sucralose, a fluidizing agent (any of light anhydrous silicic acid, hydrated silicon dioxide, and sodium stearyl fumarate) and strawberry flavor shown in each of Tables 26 and 27 were mixed using a high-speed mixer (LFS-GS-2J high-speed mixer, Fukae Powtec Co., Ltd.), and water was added to the mixture, followed by stirring granulation. Then, the granulation product was subjected to size selection in a power mill (model P-3S, Showa Kagakukikai Co., Ltd.), and the resultant was dried at 65 to 70° C. in a fluidized bed granulator (MP-01 Fluid bed dryer granulator, Powrex Corp.). After drying, a granule was obtained by size selection in a power mill (model P-3S, Showa Kagakukikai Co., Ltd.). Granulation conditions in the high-speed mixer were as follows:

### (Granulation Conditions)

Granulator: LFS-GS-2J high-speed mixer
Rotational Speed of Agitator: 333 rpm
Rotational Speed of Chopper: 2500 rpm
Acceleration in Solution Injection: 20±3.5 g/min
Moisture: 3 to 7.5% by weight
Mashing time: 1 to 2 min±5 sec

### (Temporal Stability Test of Preparation)

The produced present preparation was stored at 60° C. for 2 weeks, and the amount of increase in the compound represented by formula (II), which is a related substance, was measured.

### (Stickiness Test of Preparation)

1 g of the preparation was charged into a 4 mL brown bottle, and evaluation was made as follows: good (indicated by circle), the preparation present at the bottom fluidized when the bottle was inverted three times; fair (indicated by triangle), the preparation present in an upper part fluidized when the bottle was inverted three times; and poor (indicated by x-mark), the preparation did not fluidize when the bottle was inverted three times.

### (Results)

The amount of increase (%) in the compound represented by formula (II) in the temporal stability test of the preparations of Examples 7-15 to 7-18 and Comparative Examples 7-7 and 7-8, and the stickiness between preparations are shown in Tables 28 and 29. As a result, the amount of increase (%) in the compound represented by formula (II) in the preparations of Examples 7-15 to 7-18 was almost the same as that in the preparations of Comparative Examples 7-7 and 7-8 containing sodium stearyl fumarate, and was almost the same even when the amount of the fluidizing agent was changed.

Meanwhile, as a result of studying the stickiness of the preparations of Examples 7-15 to 7-18 and Comparative Examples 7-7 and 7-8, the preparations of Examples 7-15 to 7-18 had smaller stickiness than that of the preparations of Comparative Examples 7-7 and 7-8.

TABLE 28

| | Example 7-15 | Example 7-16 | Example 7-17 |
|---|---|---|---|
| Amount of Increase | 0.64 | 0.51 | 0.34 |
| (%) in Compound | | | |
| represented by Formula | | | |
| (II) | | | |
| Stickiness | Δ | ∘ | ∘ |

TABLE 29

| | | Comparative | Comparative |
|---|---|---|---|
| | Example 7-18 | Example 7-7 | Example 7-8 |
| Amount of Increase | 0.58 | 0.51 | 0.45 |
| (%) in Compound | | | |
| represented by Formula | | | |
| (II) | | | |
| Stickiness | ∘ | x | x |

### (6) Study on Suspending Agent

In order to study a suspending agent, the suspensibility of a preparation in water was evaluated. The present preparation having a formulation shown in Table 30 was produced by the stirring granulation method. Hypromellose (TC-5, Shin-Etsu Chemical Co., Ltd.), hydroxypropyl cellulose (HPC-L, Nippon Soda Co., Ltd.), and methyl cellulose (SM-4, Shin-Etsu Chemical Co., Ltd.) were used as the suspending agent.

TABLE 30

| | | Reference | Reference | Comparative |
|---|---|---|---|---|
| | Example 7-19 | Example 7-7 | Example 7-8 | Example 7-9 |
| | (weight mg) | (weight mg) | (weight mg) | (weight mg) |
| Compound represented by Formula | 20.0 | 20.0 | 20.0 | 20.0 |
| (I) | | | | |
| D-Mannitol | 564.0 | 564.0 | 564.0 | 564.0 |
| Hydrogenated Maltose Starch Syrup | 350.0 | 350.0 | 350.0 | 353.0 |
| (Maltitol) | | | | |
| Sodium Chloride | 30.0 | 30.0 | 30.0 | 30.0 |
| Polyvinyl Pyrrolidone | 10.0 | 10.0 | 10.0 | 10.0 |
| Hypromellose | 3.0 | — | — | — |
| Hydroxypropyl Cellulose | — | 3.0 | — | — |
| Methyl Cellulose | — | | 3.0 | — |
| Sucralose | 5.0 | 5.0 | 5.0 | 5.0 |
| Light Anhydrous Silicic Acid | 20.0 | 20.0 | 20.0 | 20.0 |
| Strawberry Flavor | 1.0 | 1.0 | 1.0 | 1.0 |
| Total | 1003.0 | 1003.0 | 1003.0 | 1003.0 |

### (Method for Producing Preparation)

A compound represented by formula (I), D-mannitol, hydrogenated maltose starch syrup (maltitol), sodium chloride and polyvinyl pyrrolidone K25 shown in Table 30 were mixed using a vertical granulator (model VG-50, Powrex Corp.), and water was added to the mixture, followed by stirring granulation. Then, the granulation product was subjected to size selection in a power mill (model P-3S, Showa Kagakukikai Co., Ltd.), and the resultant was dried at 65 to 70° C. in a fluidized bed granulator (GPGC-15&30 fluid bed dryer granulator, Powrex Corp.). After drying, size selection was performed in a power mill (model P-3S, Showa Kagakukikai Co., Ltd.). The granulation product after the size selection was mixed with sucralose, a suspending agent (any of hypromellose, hydroxypropyl cellulose, and methyl cellulose), light anhydrous silicic acid and strawberry flavor using a V-shaped mixer (130 L V type blender, manufactured by Tokuju Corp.) to obtain a granule.

### (Granulation Conditions)

Granulator: vertical granulator VG-50
Rotational Speed of Agitator: 200 rpm
Rotational Speed of Chopper: 2500 rpm
Acceleration in Solution Injection: 105±3 g/min
Moisture: 4.5 to 7.5% by weight
Mashing time: 1 to 3 min±5 sec

### (Suspensibility Test of Preparation in Water)

1 g of the present preparation was added into a stoppered container containing 9.5 mL of water, and the stoppered container was reciprocally inverted 40 times, and immediately thereafter, a liquid was collected from upper and lower parts of the container. After the completion of container inversion, the container was left at room temperature for 10 minutes, and a liquid was collected from a central part of the container. The concentration of the compound represented by formula (I) in the collected liquids was measured.

(Method for Measuring Compound represented by Formula (I))

The amount of the compound represented by formula (I) was measured by liquid chromatography by employing the following method and conditions:
Detector: ultraviolet absorptiometer (measurement wavelength: 260 nm)
Column: ACQUITY UPLC BEH C18 1.7 μm, 2.1×50 mm (Waters Corp.)
Column temperature: constant temperature around 35° C.
Mobile Phase A: 0.1% trifluoroacetic acid/0.2 mM EDTA solution, Mobile Phase B: acetonitrile
Delivery of mobile phase: controlled for a concentration gradient with a mixing ratio between the mobile phase A and the mobile phase B changed as shown in Table 31

TABLE 31

| | Mobile Phase | Mobile Phase |
|---|---|---|
| Time after Injection (min) | A (vol %) | B (vol %) |
| 0-2.3 | 62 | 38 |
| 2.3-3 | 62 → 20 | 38 → 80 |
| 3-4 | 20 | 80 |

Flow rate: about 0.6 mL/min
Injection amount: 4 μL
Sample cooler temperature: about 5° C.
Washing solution for autoinjector: acetonitrile
Range of area measurement: 8 minutes after injection of sample solution
Equation for calculating amount of compound represented by formula (I):

Amount of compound represented by formula (I)(%)=MS/C×A_{T}/A_{S}×100
MS: weighed amount (mg)
C: labeled amount in preparation (mg/mL)
A_{S}: peak area obtained from standard solution
A_{T}: peak area obtained from sample solution

### (Evaluation of Suspensibility in Water)

The suspensibility of the preparation was evaluated according to the following equation: Ratio (%) of amount of compound represented by formula (I) in suspension at central position of container after 10 minutes from container inversion=(Concentration of compound represented by formula (I) in suspension at central position of container after 10 minutes from container inversion/Concentration of compound represented by formula (I) in suspension at central position of container immediately after container inversion)×100(%)

### (Results)

The suspensibility in water of the preparations of Example 7-19, Reference Examples 7-7 and 7-8 and Comparative Example 7-9 is shown in Table 32. As a result, the ratio of the amount of the compound represented by formula (I) in the suspensions of Example 7-19 and Reference Examples 7-7 and 7-8 was higher than that in the suspension of Comparative Example 7-9 containing no suspending agent. Particularly, the preparation of Example 7-19 containing hypromellose had a high ratio of the amount of the compound represented by formula (I) in the suspension and had good suspensibility in water.

TABLE 32

| | | Reference | Reference | Comparative |
|---|---|---|---|---|
| | Example 7-19 | Example 7-7 | Example 7-8 | Example 7-9 |
| Ratio (%) of amount of | 95.1 | 93.0 | 92.9 | 65.8 |
| compound represented | | | | |
| by formula (I) in | | | | |
| suspension at central | | | | |
| position of container | | | | |
| after 10 minutes from | | | | |
| container inversion | | | | |

### (7) Study on Lubricant

In order to study a lubricant, an angle of repose was evaluated as an index for fluidity of a preparation. A preparation having a formulation shown in Table 33 was produced by the stirring granulation method. Talc (Merck KGaA, LUB) was used as the lubricant.

TABLE 33

| | | Comparative |
|---|---|---|
| | Example 7-20 | Example 7-10 |
| | (weight mg) | (weight mg) |
| Compound represented by Formula | 20.0 | 20.0 |
| (I) | | |
| D-Mannitol | 560.0 | 561.0 |
| Powdered Hydrogenated Maltose | 350.0 | 350.0 |
| Starch Syrup (Maltitol) | | |
| Sodium Chloride | 30.0 | 30.0 |
| Polyvinyl Pyrrolidone | 10.0 | 10.0 |
| Hypromellose | 3.0 | 3.0 |
| Sucralose | 5.0 | 5.0 |
| Light Anhydrous Silicic Acid | 20.0 | 20.0 |
| Talc | 1.0 | - |
| Strawberry Flavor | 1.0 | 1.0 |
| Total | 1000.0 | 1000.0 |

### (Method for Producing Preparation)

A compound represented by formula (I), D-mannitol, hydrogenated maltose starch syrup (maltitol), sodium chloride, polyvinyl pyrrolidone K25, and hypromellose shown in Table 33 were mixed using a vertical granulator (model FM-VG50, Powrex Corp.), and water was added to the mixture, followed by stirring granulation. Then, the granulation product was subjected to size selection in a power mill (model P-3S, Showa Kagakukikai Co., Ltd.), and the resultant was dried at 65 to 70° C. in a fluidized bed granulator (GPGC-15&30 fluid bed dryer granulator, Powrex Corp.). After drying, size selection was performed in a power mill (model P-3S, Showa Kagakukikai Co., Ltd.). The granulation product after the size selection was mixed with talc, sucralose, light anhydrous silicic acid and strawberry flavor using a V-shaped mixer (130 L V type blender, Tokuju Corp.) to obtain a granule.

### (Granulation Conditions)

Granulator: vertical granulator VG-50
Rotational Speed of Agitator: 200 rpm
Rotational Speed of Chopper: 2500 rpm
Acceleration in Solution Injection: 105±3 g/min
Moisture: 4.5 to 7.5% by weight
Mashing time: 1 to 3 min±5 sec

### (Measurement of Angle of Repose of Preparation)

The angle of repose of the produced preparation was measured using a powder tester (Hosokawa Micron Group) under the following conditions:
Operation time: 170 sec, Slow down: 10 sec, Amplitude: 1.5 mm

### (Results)

The angle of repose of the preparations of Example 7-20 and Comparative Example 7-10 is shown in Table 34. As a result, the preparation of Example 7-20 containing talc had a smaller angle of repose than that of the preparation of Comparative Example 7-10 containing no talc, demonstrating that the fluidity of the preparation can be enhanced by containing talc.

TABLE 34

| | | Comparative |
|---|---|---|
| | Example 7-20 | Example 7-10 |
| Angle of Repose (°) | 33.7 | 36.2 |

### (8) Measurement of Release Rate

The preparation of Example 7-20 shown in Table 33 was stored at 60° C. for 2 weeks and at 40° C. and 75% relative humidity for 2 weeks, and the release rate of the compound represented by formula (I) was measured.

### (Dissolution Property Test of Preparation)

The produced preparation was stored at 60° C. for 2 weeks and at 40° C. and 75% relative humidity for 2 weeks, and the release rate of the compound represented by formula (I) was measured by the second method of Dissolution Test described in the Japanese Pharmacopoeia (paddle method). The fluid used in the method of Dissolution Test was the dissolution test second fluid (containing 1% Tween 20), and the rotational speed of the paddle was set to 50 rpm.

### (Results)

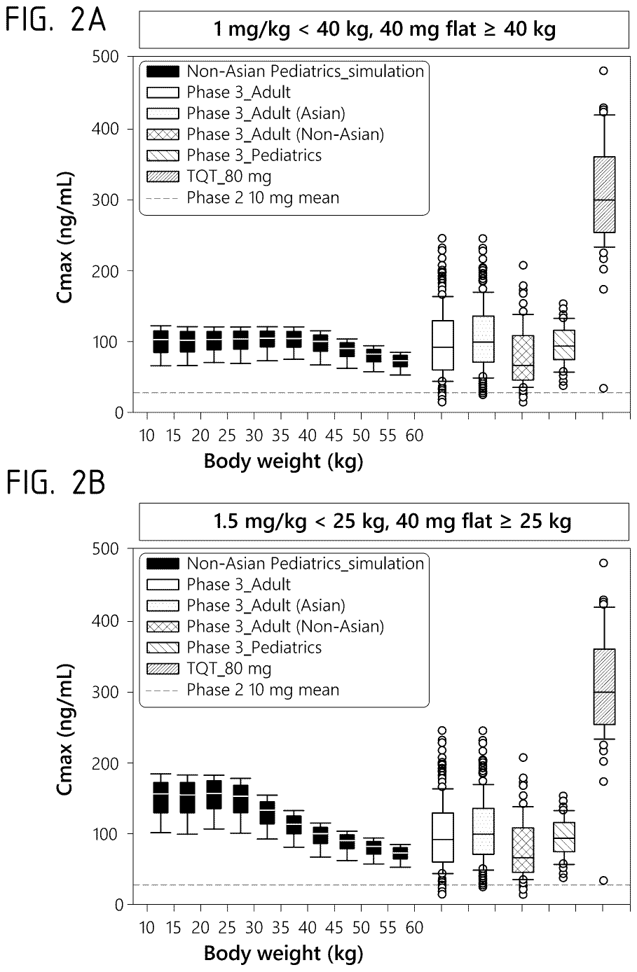

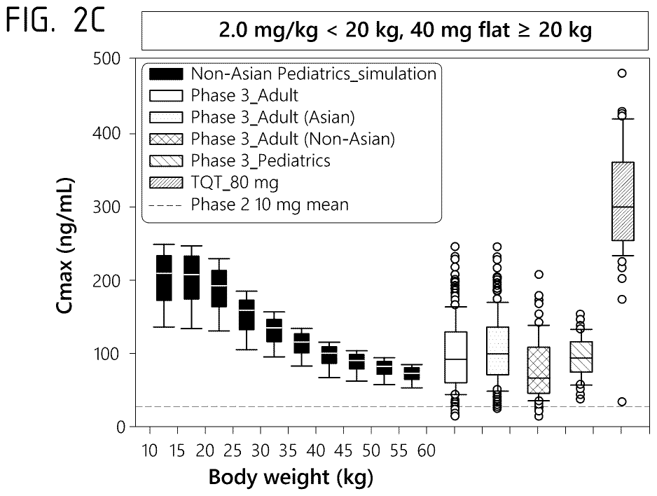

As shown in FIGS. 2A-2C, the release rate from the preparation of Example 7-20 after storage at 60° C. for 2 weeks and after storage at 40° C. and 75% relative humidity for 2 weeks hardly differed from the release rate from the preparation immediately after preparation.

### (9) Preparation Having Different Composition Ratio

Example 7-21 shown in Tables 34 was prepared in the same manner of Example 7-20 by the stirring granulation method.

TABLE 35

| | Example 7-21 |
|---|---|
| | (weight mg) |
| Compound represented by Formula (I) | 40.0 |
| D-Mannitol | 540.0 |
| Powdered Hydrogenated Maltose Starch Syrup | 350.0 |
| (Maltitol) | |
| Sodium Chloride | 30.0 |
| Polyvinyl Pyrrolidone | 10.0 |
| Hypromellose | 3.0 |
| Sucralose | 5.0 |
| Light Anhydrous Silicic Acid | 20.0 |
| Talc | 1.0 |
| Strawberry Flavor | 1.0 |
| Total | 1000.0 |

B. Preparation of Granules which are Optimized for the Preparation of an Oral Suspension

Granules which are optimized for the preparation of an oral suspension have been prepared. The granulated powder was manufactured via a standard wet granulation process. The detailed composition of the granules for oral suspension is shown Table 1 and the rationale for use of the excipients is provided. The excipients and their amounts are known to be suitable for the intended paediatric populations from 0 to <18 years of age. The granulae can easily be reconstituted with water. More specifically, 2 g granulae, which contain 40 mg of baloxavir marboxil (nominal) can be reconstituted with 20 mL water, which corresponds to a final concentration of 2 mg of the compound/mL.

The detailed composition of the granules for oral suspension is shown Table 36.

TABLE 36
Components and Composition of Baloxavir
marboxil Granules for Oral Suspension

| | Nominal | Concentration | | |
|---|---|---|---|---|
| | amount | in Granule | | |
| Component | (mg/bottle) | (%) | Function | Quality Standard |
| Baloxavir Marboxil | 40 | 2 | Active | In-house standard |
| | | | ingredient | |
| Mannitol | 1120 | 56 | Diluent | Ph. Eur./USP/JP |
| Maltitol | 700 | 35 | Diluent | Ph. Eur./NF/JPE |
| Sodium Chloride | 60 | 3 | Taste | Ph. Eur./USP/JP |
| | | | masking | |
| | | | agent | |
| Hypromellose | 6 | 0.3 | Dispersant | Ph. Eur./USP/JP |
| Povidone (K value: | 20 | 1 | Binder | Ph. Eur./USP/JP |
| 25) | | | | |
| Silica, Colloidal | 40 | 2 | Fluidizer | Ph. Eur./NF/JP |
| Anhydrous | | | | |
| Sucralose | 10 | 0.5 | Sweetener | Ph. Eur./NF/JPE |
| Talc | 2 | 0.1 | Lubricant | Ph. Eur./USP/JP |
| Strawberry Flavour | 2 | 0.1 | Flavour | In-house standard |
| Purified Water^{a} | — | — | Vehicle | Ph. Eur./USP/JP |
| Total Weight^{b} | 2,000 | 100 | — | — |

^{a}Purified water is removed during manufacturing process.
^{b}An overfill of, e.g. 0.13 g of granules is applied to obtain the targeted maximum extractable volume of 20 mL after reconstitution; fill weight may be adjusted based on assay value for bulk granules.

Bitter taste has been reported in adult clinical studies with baloxavir marboxil and several excipients have been included in the formulation to mask the bitter taste and ensure palatability, such as sodium chloride, sucralose and strawberry flavour. Thus, the granulae provided herewith have the advantages that they are to be administered in the form of an oral suspension and that the bitter taste of the active compound is masked. Accordingly, these granulae improve acceptance of the compound in paediatric patients, which contributes to the achievement of the therapeutic effect.

### Example 7: Global Phase III Study Investigating One-Dose Baloxavir Marboxil (XOFLUZA) in Children with the Flu

### Methods

miniSTONE-2 was a phase III, global multicenter, randomized, double-blind, active-controlled study in otherwise healthy paediatric patients with influenza, conducted during the 2018/19 season mainly in the US. The study evaluated the safety (primary objective), pharmacokinetics (PK) and efficacy (secondary objective) of one-dose of baloxavir marboxil (granular formulation for suspension) in otherwise healthy children aged 1 to less than 12 years with influenza. More specifically, the effect of baloxavir marboxil was compared to the effect of oseltamivir. The influenza infection was confirmed by a rapid influenza diagnostic test and displaying influenza-like symptoms (a temperature of 38° C. or over, and one or more respiratory symptoms).

Patients were randomized 2:1 to receive either a weight-based single oral dose of baloxavir marboxil or standard oral dose of oseltamivir (twice-daily dosing for five days). More specifically, participants enrolled in the study were recruited in parallel into two cohorts: patients aged five to less than 12 years and patients aged one to less than 5 years. Patients in both cohorts were randomly assigned to receive one-dose of baloxavir marboxil (2 mg/kg for patients under 20 kg or 40 mg for patients 20 kg or over) or oseltamivir twice a day over five days (dosing according to body weight).

The primary endpoint was the proportion of patients with adverse events or severe adverse events up to study day 29. Secondary endpoints include pharmacokinetics (PK), time to alleviation of influenza signs and symptoms, and duration of symptoms, including fever and time to cessation of viral shedding by virus titer for virology.

### Results in Summary

This study investigated the safety (primary objective), pharmacokinetics and efficacy of a single dose of baloxavir marboxil in otherwise healthy children aged 1 to <12 years with influenza. The study showed that baloxavir marboxil (XOFLUZA), given as a new oral suspension, is a well-tolerated and effective potential treatment for the flu in otherwise healthy children aged one to less than 12 years.

The obtained results can be summarized as follows.
Baloxavir was well tolerated and no new safety signals were identified
   no SAEs
No relevant differences in demographics or clinical baseline characteristics were noticed between baloxavir and oseltamivir groups
   median age 6 years; 53% female; 85% Caucasian; no relevant differences observed between baloxavir and oseltamivir groups
Pharmacokinetic data
   initial ‘lead in’ PK indicated baloxavir exposure to be consistent with adults and adolescents
Baloxavir showed comparable efficacy compared with oseltamivir in Time To Alleviation of Influenza Signs and Symptoms (TASS) endpoint
   TASS uses cough, nasal symptoms, return to daycare/school/normal activities (from parent/carer questionnaire) and fever
   TASS: baloxavir 138 hrs (CI 116.6, 163.2); oseltamivir 150 hrs (CI 115.0, 165.7)
   TASS was an exploratory endpoint and did not undergo statistical testing. The almost identical confidence intervals indicate comparable efficacy between treatment
Clear difference in Time To Cessation of Viral Shedding
   There was a clear difference in the median Time to Cessation of Viral Shedding between baloxavir (24 hrs) and Oseltamivir (76 hrs); delta 56 hrs. These data continue to suggest that baloxavir-treated patients are no longer infective after a median time of 1 day compared to 3 days in oseltamivir-treated patients. This may be of significance to the reduction of onward transmission of influenza.

### Results in Detail:

The study assessed baloxavir marboxil versus an active comparator (oseltamivir) in children aged between one and less than 12 years with the flu.

Of the 176 paediatric patients recruited, 124 formed the ITTi population (baloxavir marboxil, n=81 vs oseltamivir, n=43), 89.7% of which had an influenza A infection (65.5% H3N2, 24.1% H1N1). No SAEs, deaths or adverse events of special interest were observed and the safety profile of baloxavir marboxil was consistent with that observed in clinical studies to date. The median time to alleviation of influenza signs and symptoms observed in the BXM group (138 hours [95% CI; 116.6, 163.2]) was comparable to the oseltamivir group (150 hours [95% CI; 115.0, 165.7]). Consistent with previous phase III studies, there was a clear difference in the median time to cessation of viral shedding between baloxavir marboxil (24.2 hours [95% CI; 23.5, 24.6]) and oseltamivir (75.8 hours [95% CI; 68.9, 97.8]).

Thus, the phase III miniSTONE-2 study met its primary endpoint, demonstrating that baloxavir marboxil (XOFLUZA) is well-tolerated in children with the flu. As described above, the study also showed that baloxavir marboxil is comparable to oseltamivir—a proven effective treatment for children with the flu—at reducing the duration of flu symptoms, including fever.

### CONCLUSION

A single, oral dose of baloxavir marboxil was well tolerated and effective for the treatment of influenza in otherwise healthy paediatric patients aged between 1 and <12 years. The MINISTONE-2 study showed that baloxavir marboxil (XOFLUZA), given as a new oral suspension, is a well-tolerated and effective potential treatment for the flu in otherwise healthy children aged one to less than 12 years.

### Example 8: Biological Sequences

The present invention refers to the following nucleotide and amino acid sequences:

SEQ ID NO: 1:
Influenza A virus (A/WSN/1933(H1N1)): GenBank:
X17336.1, comprising the I38T mutation. The I38T
mutation is underlined and shown in bold face.
MEDFVRQCFNPMIVELAEKAMKEYGEDLKIETNKFAATCTHLEVCFMYSD
FHFIDEQGESIVVELGDPNALLKHRFEIIEGRDRTIAWTVINSICNTTGA
EKPKFLPDLYDYKKNRFIEIGVTRREVHIYYLEKANKIKSEKTHIHIFSF
TGEEMATKADYTLDEESRARIKTRLFTIRQEMASRGLWDSFRQSERGEET
IEERFEITGTMRKLADQSLPPNFSSLENFRAYVDGFEPNGYIEGKLSQMS
KEVNARIEPFLKSTPRPLRLPDGPPCSQRSKFLLMDALKLSIEDPSHEGE
GIPLYDAIKCMRTFFGWKEPNVVKPHEKGINPNYLLSWKQVLAELQDIEN
EEKIPRTKNMKKTSQLKWALGENMAPEKVDFDDCKDVGDLKQYDSDEPEL
RSLASWIQNEFNKACELTDSSWIELDEIGEDAAPIEHIASMRRNYFTAEV
SHCRATEYIMKGVYINTALLNASCAAMDDFQLIPMISKCRTKEGRRKTNL
YGFIIKGRSHLRNDTDVVNFVSMEFSLTDPRLEPHKWEKYCVLEVGDMLL
RSAIGHVSRPMFLYVRTNGTSKIKMKWGMEMRRCLLQSLQQIESMIEAES
SVKEKDMTKEFFENKSETWPVGESPKGVEEGSIGKVCRTLLAKSVFNSLY
ASPQLEGFSAESRKLLLIVQALRDNLEPGTFDLGGLYEAIEECLINDPWV
LLNASWFNSFLTHALR

SEQ ID NO:2: Sequence fraction of the influenza A virus (A/WSN/1933(H1N1)): GenBank: X17336.1, comprising the I38T mutation. The I38T mutation is underlined and shown in bold face.

FAATCTH

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for treating an influenza virus infection, wherein said method comprises administering an effective amount of a compound to a patient having an influenza virus infection, wherein the compound has one of the following formulae (I) and (II): 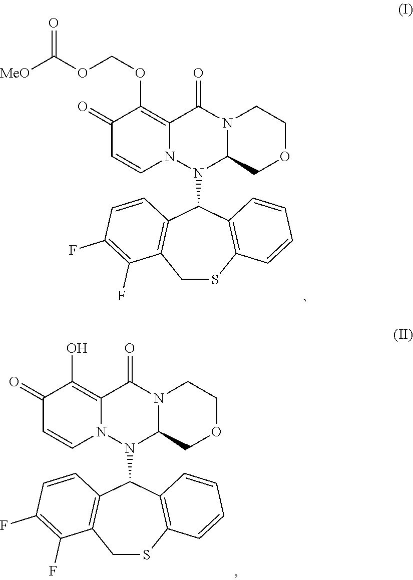 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or is a pharmaceutically acceptable salt thereof,
and wherein the following dosage is used:
(i) in a patient that is younger than 1 year:
(a) if the patient is younger than 4 weeks, then the effective amount is 0.8-1.2 mg/kg body weight, preferably about 1 mg/kg body weight;
(b) if the patient is 4 weeks or older but younger than 3 months, then the effective amount is 0.8-1.2 mg/kg body weight, preferably about 1 mg/kg body weight;
(c) if the patient is 3 months or older but younger than 12 months, then the effective amount is 1.8-2.2 mg/kg body weight, preferably about 2 mg/kg body weight;
(ii) in a patient that is 1 year or older but younger than 12 years:
(a) if the patient has a body weight of less than 20 kg, then the effective amount is 1.8-2.2 mg/kg body weight, preferably about 2 mg/kg body weight; or
(b) if the patient has a body weight of 20 kg or more, then the effective amount is 35-45 mg, preferably about 40 mg.

2. The method of claim 1, wherein the patient is white.

3. The method of claim 1, wherein the patient does not have an Asian ethnicity.

4. The method of claim 1, wherein the compound is administered in the form of a suspension of granules.

5. The method of claim 1, wherein the compound is orally administered.

6. The method of claim 1, wherein the patient is 1 year old or older but younger than 5 years.

7. The method of claim 1, wherein the patient is 5 years old or older but younger than 12 years.

8. The method of claim 1, wherein the patient has a body weight which is less than 40 kg.

9. The method of claim 1, wherein the patient is healthy except for the influenza virus infection.

10. The method of claim 1, wherein the patient is diagnosed as having an influenza virus infection:
(a) due to the presence of fever of 38° C. or more (tympanic temperature); and at least one respiratory symptom, preferably cough and/or nasal congestion; and/or
(b) by using an influenza test kit.

11. The method of claim 1, wherein the influenza virus is a type A influenza virus.

12. The method of claim 1, wherein the compound is administered within 96 hours from the time of symptom onset, preferably within 48 hours from the time of symptom onset.

13. The method of claim 12, wherein the symptom onset is the time point of the onset of at least one systemic symptom and/or at least one respiratory symptom.

14. The method of claim 13, wherein the at least one systemic symptom is at least one symptom selected from headache, feverishness, chills, muscular pain, joint pain, and fatigue.

15. The method of claim 13, wherein the at least one respiratory symptom is at least one symptom selected from coughing, sore throat, and nasal congestion.

16. The method of claim 1, wherein the treated patient to whom the compound has been administered has a decreased virological activity as compared to an untreated patient to whom the compound has not been administered.

17. The method of claim 16, wherein the virological activity is measured by:
(i) determination of the time to cessation of viral shedding;
(ii) determination of the influenza virus titer; and/or
(iii) determination the amount of virus RNA.

18. The method of claim 17, wherein the duration of influenza virus shedding is measured as time to shedding cassation following symptom onset.

19. The method of claim 17, wherein the amount of virus RNA is measured by using reverse transcriptase-polymerase chain reaction (RT-PCR).

20. The method of claim 1, wherein the compound reduces the time to alleviation of influenza signs and symptoms (TASS) by at least 6 hours, preferably by at least about 12 hours as compared to an untreated patient to whom the compound has not been administered.

21. The method of claim 1, wherein the time from diagnosis of the influenza virus infection until recovery is decreased in the treated patient to whom the compound has been administered as compared to an untreated patient to whom the compound has not been administered.

22. The method of claim 20, wherein the patient has recovered when at least one of the following recovery criteria is met and remains met for at least 21.5 hours:
(i) return to afebrile state (tympanic temperature ≤37.2° C.);
(ii) a score of 0 (no problem) or 1 (minor problem) for cough and nasal symptoms as specified in items 14 and 15 of the Canadian Acute Respiratory Illness and Flu Scale (CARIFS), preferably a score of 0 (no problem) or 1 (minor problem) for all 18 symptoms specified in the (CARIFS);
(iii) cessation of viral shedding; and/or
(iv) return to normal health and activity.

23. The method of claim 22, wherein return to normal health and activity is achieved if the patient is able to return to day care or school, and/or to resume his or her normal daily activity in the same way as performed prior to developing the influenza virus infection.

24. The method of claim 16, wherein the untreated patient has been administered with oseltamivir.

25. The method of claim 1, wherein the administration of the compound prevents the occurrence of an influenza-related complication.

26. The method of claim 25, wherein the influenza-related complication is at least one of the complications selected from the group consisting of radiologically confirmed pneumonia, bronchitis, sinusitis, otitis media, encephalitis/encephalopathy, febrile seizures, and myositis.

27. The method of claim 1, wherein death of the patient caused by the influenza virus infection is prevented by the administration of the compound.

28. The method of claim 1, wherein the requirement of antibiotics is prevented by the administration of the compound.

29. The method of claim 1, wherein the compound has the formula (I).


SEQUENCE LISTING (2 sequences)

SEQ ID NO: 1 - protein, 716 aa, unidentified
  REGION 1..716: note = GenBank: X17336.1, comprising the I38T mutation
MEDFVRQCFNPMIVELAEKAMKEYGEDLKIETNKFAATCTHLEVCFMYSDFHFIDEQGESIVVELGDPNALLKHRFEIIEGRDRTIAWTVINSICNTTGAEKPKFLPDLYDYKKNRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLENFRAYVDGFEPNGYIEGKLSQMSKEVNARIEPFLKSTPRPLRLPDGPPCSQRSKFLLMDALKLSIEDPSHEGEGIPLYDAIKCMRTFFGWKEPNVVKPHEKGINPNYLLSWKQVLAELQDIENEEKIPRTKNMKKTSQLKWALGENMAPEKVDFDDCKDVGDLKQYDSDEPELRSLASWIQNEFNKACELTDSSWIELDEIGEDAAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEVGDMLLRSAIGHVSRPMFLYVRTNGTSKIKMKWGMEMRRCLLQSLQQIESMIEAESSVKEKDMTKEFFENKSETWPVGESPKGVEEGSIGKVCRTLLAKSVFNSLYASPQLEGFSAESRKLLLIVQALRDNLEPGTFDLGGLYEAIEECLINDPWVLLNASWFNSFLTHALR

SEQ ID NO: 2 - protein, 7 aa, unidentified
  REGION 1..7: note = Sequence Fraction GenBank: X17336.1, comprising the I38T mutation
FAATCTH